(19) United States
(12) Patent Application Publication
(10) Pub. No.: US 2021/0292437 A1
(43) Pub. Date: Sep. 23, 2021

(54) CHIMERIC QUIESCIN SULFHYDRYL OXIDASE (QSOX1) ANTIBODIES AND USES OF SAME

(71) Applicant: Yeda Research and Development Co. Ltd., Rehovot (IL)

(72) Inventors: Deborah FASS, Rehovot (IL); Sarel FLEISHMAN, Rehovot (IL); Shira WARSZAWSKI, Rehovot (IL); Iris GROSSMAN, Rehovot (IL); Lev KHMELNITSKY, Rehovot (IL)

(73) Assignee: Yeda Research and Development Co. Ltd., Rehovot (IL)

(21) Appl. No.: 17/267,049

(22) PCT Filed: Aug. 14, 2019
(86) PCT No.: PCT/IL2019/050914

§ 371 Date: Feb. 9, 2021

(30) Foreign Application Priority Data
Aug. 14, 2018 (IL) 261156

(51) Int. Cl. C07K 16/40

(52) U.S. Cl. CPC C07K 16/40; C07K 2317/565; C07K 2317/24

(57) ABSTRACT

Antibodies for binding QSOX1 are provided. Also provided are use of these antibodies.

### RELATED APPLICATION/S

This application claims the benefit of priority from Israel Patent Application No. 261156 filed Aug. 14, 2018, which is hereby incorporated by reference in its entirety.

### SEQUENCE LISTING STATEMENT

The ASCII file, entitled 78364 Sequence Listing.txt, created on 6 Aug. 2019, comprising 90,609 bytes, submitted concurrently with the filing of this application is incorporated herein by reference.

### FIELD AND BACKGROUND OF THE INVENTION

The present invention, in some embodiments thereof, relates to chimeric Quiescin Sulfhydryl Oxidase (QSOX1) antibodies and uses of same.

Monoclonal antibody therapy has become an integral part of cancer diagnostics and treatment. The success of antibody-based therapy stems from the high specificity and affinity that antibodies offer compared to other anti-tumor agents. In addition, tumors express on their cell-surfaces many potential targets for antibody therapeutics. Antibodies supplied extracellularly can both neutralize the function of their cell-surface antigen and recruit the immune system for a more extensive anti-tumor response. Recently, the realization that tumor stroma has a major role in supporting tumor development and metastasis inspired antibody-based cancer therapies targeting extracellular matrix (ECM) components in addition to targeting tumor cells directly. Examples for such agents are antibodies that affect the extracellular glycoprotein tenascin or fibroblast activation protein, found in stromal fibroblasts of most human carcinomas.

ECM proteins are good candidates for antibody therapy because they are both accessible and abundant in most tissues, making the same ECM components a target in various cancers. A major component of the ECM that is over-expressed, reorganized, and cross-linked in tumorigenesis is collagen. Collagen cross-linking by the enzyme lysyl oxidase (LOX) is increased in several cancers and contributes to matrix stiffening, thereby promoting cell adhesion and migration. Inhibitors of LOX activity, including monoclonal antibodies, significantly inhibited tumor growth and metastasis in gastric carcinoma. Laminin is another abundant scaffolding ECM protein that interacts with integrins to mediate cell adhesion and migration, a requirement for metastasis. Indeed, laminin is over-expressed in various cancers, and its chain isotypes serve as tumor biomarkers Like collagen cross-linking and integrin blocking, laminin incorporation into the matrix may serve as a complementary target for antibody-based cancer therapeutics.

Fass D. et al. have shown that laminin incorporation into the ECM is affected by the disulfide catalyst Quiescin sulfhydryl oxidase 1 (QSOX1) [Ilani, T. et al. (2013) Science 341: 74-76]. The enzyme QSOX1 is a fusion of two thioredoxin (Trx) domains and an Erv-fold sulfhydryl oxidase module. QSOX1 contains two CXXC motifs as redox-active sites that cooperate to relay electrons from reduced thiols of substrate proteins to molecular oxygen. Mechanistically, after oxidizing the substrate, the Trx active site transfers two electrons to the Erv CXXC motif through an inter-domain disulfide intermediate. The electrons proceed to the adjacent flavin adenine dinucleotide cofactor, which in turn reduces oxygen to hydrogen peroxide, leaving QSOX1 oxidized and ready for another catalytic cycle. Unlike other disulfide catalysts, QSOX1 is localized downstream of the endoplasmic reticulum (ER). It is found in the Golgi apparatus and secreted from quiescent fibroblasts into the ECM, where it affects ECM composition and especially laminin incorporation [Ilani T. et al. (2013), supra]. Specifically, QSOX1 affects the incorporation of laminin isoforms that contain an α4 chain [Ilani T. et al. (2013), supra], a known marker for tumor progression. Together with the over-production of QSOX1 in various adenocarcinomas and associated stroma, these findings point to a possible role of QSOX1 in stimulating tumor cell migration via laminin incorporation.

The multi-step catalytic cycle of QSOX1 implies that obscuring any one of several sites on the protein by interaction with antibody may accomplish inhibition. Fass D. and co-workers have developed an inhibitory monoclonal antibody, MAb492.1, which blocks substrate access to the Trx CXXC redox-active site of human QSOX1 (HsQSOX1) [Grossman I. et al. (2013) J. Mol. Biol. 425: 4366-4378]. MAb492.1 efficiently inhibited HsQSOX1 activity, and consequently inhibited adhesion and migration of cancer cells to and through fibroblasts from corresponding tissues [Ilani T. et al. (2013), supra]. Accordingly, MAb492.1 may serve as an anti-metastatic drug in antibody-based cancer therapy.

Additional background art includes:

WO2013/132495

### SUMMARY OF THE INVENTION

According to an aspect there is provided an isolated antibody comprising an amino acid sequence of a light chain (vL) as set forth in SEQ ID NO: 1 and a heavy chain (vH) as set forth in SEQ ID NO: 2, wherein at least one of the vL and the vH comprises at least one amino acid substitution selected from the group consisting of:

| | |
|---|---|
| 34L | AS |
| 43L | SAPT |
| 46L | LV |
| 55L | YHPV |
| 89L | QH |
| 96L | LF |
| 37H | VF |
| 44H | GAKR |
| 45H | LF |
| 58H | DE |
| 94H | FY |
| 104H | SA |
| 105H | FLY |
| 106H | AENPQ |

wherein the antibody binds QSOX1.

According to an aspect there is provided an isolated antibody comprising an antigen recognition domain comprising complementarity-determining regions as set forth in GFSLTGYG (CDRHI) (SEQ ID NO: 83), IWGDGRT (CDRH2) (SEQ ID NO: 84) and ASDYYGSGX₁X₂X₃Y, (CDRH3) (SEQ ID NO: 97), QDVSTA (CDRLI) (SEQ ID NO: 86), SAS (CDRL2) (SEQ ID NO: 87) and QQHYSIPX₄T (CDRL3) (SEQ ID NO: 98), wherein Xi, X₂, X₃ and X₄ is any amino acid, wherein the antibody binds QSOX1, wherein when the antibody comprises CDRH3 set forth in SEQ ID NO: 85 the CDRL3 is not as set forth in SEQ ID NO: 88.

According to some embodiments, the X₁ is selected from the group consisting of S and A.

According to some embodiments, the X₂ is selected from the group consisting of F, L and Y.

According to some embodiments, the X₃ is selected from the group consisting of A, E, N, P and Q.

According to some embodiments, the X₄ is F.

According to some embodiments, the CDRH3 is selected from the group consisting of ASDYYGSGALEY (SEQ ID NO: 99), ASDYYGSGALAY (SEQ ID NO: 100), ASDYYGSGAFEY (SEQ ID NO: 101), ASDYYGSGALEY (SEQ ID NO: 102), ASDYYGSGSFPY (SEQ ID NO: 103), ASDYYGSGSFNY (SEQ ID NO: 104), ASDYYGSGALEY (SEQ ID NO: 105), ASDYYGSGSLEY (SEQ ID NO: 106), ASDYYGSGSYQY (SEQ ID NO: 107), ASDYYGSGALQY (SEQ ID NO: 108), ASDYYGSGSLAY (SEQ ID NO: 109), ASDYYGSGAFQY (SEQ ID NO: 110), ASDYYGSGSFQY (SEQ ID NO: 111), ASDYYGSGSLAY (SEQ ID NO: 112), ASDYYGSGAYEY (SEQ ID NO: 113), ASDYYGSGSYQY (SEQ ID NO: 114), ASDYYGSGSYQY (SEQ ID NO: 115), ASDYYGSGALAY (SEQ ID NO: 116), ASDYYGSGALAY (SEQ ID NO: 117) and ASDYYGSGSYAY (SEQ ID NO: 118).

According to some embodiments, the at least one amino acid substitution comprises at least two amino acid substitutions.

According to some embodiments, the at least one amino acid substitution comprises at least four amino acid substitutions.

According to some embodiments, the at least one amino acid substitution comprises at least five amino acid substitutions.

According to some embodiments, the at least two amino acid substitutions are at the vL and vH.

According to some embodiments, the at least one amino acid substitution is at the position 43L (“L” refers to the light chain).

According to some embodiments, the at least one amino acid substitution is at the position 55L.

According to some embodiments, the at least one amino acid substitution is at the position 43L and 55L.

According to some embodiments, the antibody exhibits an increased expression yield when expressed in HEK293F cells as determined by Western blot and at least about the same QSOX1 inhibitory activity as determined by sulfhydryl oxidase assay as that of an antibody designated m492.1.

According to some embodiments, the antibody is as set forth in SEQ ID NOs: 8, 48 and 38, 78.

According to some embodiments, the antibody inhibits QSOX1 activity in mediating laminin incorporation in the basement membrane.

According to some embodiments, the activity is assayed by at least one of an immunofluorescence (IF) staining assay of an extracellular matrix or a Western blot assay detecting for soluble laminin.

According to some embodiments, the antibody is immobilized to a solid support.

According to some embodiments, the antibody is attached to a detectable moiety.

According to an aspect there is provided a pharmaceutical composition comprising as an active ingredient the antibody as described herein and a pharmaceutically acceptable carrier.

According to an aspect there is provided a method for preventing or treating a laminin-associated disease or condition in a subject in need thereof, the method comprising administering to the subject a therapeutically effective amount of the pharmaceutical composition as described herein.

According to some embodiments, the method further comprises administering to the subject a chemotherapeutic agent.

According to some embodiments, the antibody is for use in preventing or treating a laminin-associated disease or condition in a subject in need thereof.

According to some embodiments, the antibody for use further comprises the use of a chemotherapeutic agent.

According to an aspect there is provided an article of manufacture comprising the antibody as described herein being packaged in a packaging material and identified in print, in or on the packaging material for use in the treatment of a laminin-associated disease or condition.

According to some embodiments, the article of manufacture further comprises a chemotherapeutic agent.

According to some embodiments, the laminin-associated disease or condition is a tumor.

According to some embodiments, the tumor is a metastasizing solid tumor.

According to some embodiments, the tumor is an adenocarcinoma.

According to some embodiments, the tumor is a cancer selected from the group consisting of a prostate cancer, a lung cancer, a breast cancer, a cervical cancer, an urachus cancer, a vaginal cancer, a colon cancer, an esophagus cancer, a pancreatic cancer, a throat cancer, a stomach cancer and a myeloid leukemia.

According to some embodiments, the laminin-associated disease or condition is associated with fibrosis.

According to an aspect there is provided an isolated polynucleotide comprising a nucleic acid sequence encoding the antibody as described herein.

According to an aspect there is provided a cell culture comprising cells expressing the isolated polynucleotide as described herein.

According to an aspect there is provided a method of producing an antibody to QSOX1, the method comprising:

(a) culturing cells as described herein under conditions which allow for expression of the vH and/or vL chains;
(b) recovering the vH and/or vL chains from the cells.

According to some embodiments, the cells are HEK 293F cells.

According to some embodiments, the method comprises subjecting the vH and vL chains to refolding.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-D show an increase in antibody expression yields following computational design. (FIG. 1A) Dot blot analysis shows no detectable expression for h492.1 in HEK293F cells, whereas all 20 designs show detectable levels of expression. (FIG. 1B) Relative expression levels of the 20 designs using Western blot analysis. h492.1^{des3}, and h492.1^{des18} showed high expression, and were selected for further analysis. (FIG. 1C) QSOX1 inhibitory activity assay using the parental 492.1 antibody and two designs. The inhibitory activity was measured for each antibody in a sulfhydryl oxidase assay using a physiological concentration of QSOX1 (25 nM). h492.1^{des18} showed comparable inhibitory activity relative to the parental antibody, with only a slight decrease when provided at sub-stoichiometric amounts (10 nM). (FIG. 1D) Structural context of mutations in h492.1^{des18} (color) relative to the experimental structure of 492.1 (gray). Spheres indicate the locations of the mutations, and thumbnail shows two of the four designed mutations, which improve interchain packing and rigidify the backbone at the vL-vH interface according to the model structure.

FIG. 2 is a dot blot of exemplary designs that are expressible in HEK293F cells, according to some embodiments of the invention. Suspension-adapted human embryonic kidney cells, HEK293F, were transiently transfected in duplicates (plates 1 and 2) with plasmids encoding the 20 designs and the wild-type chimera h492.1 (wt). Additionally, three mutants of the h492.1 light chain (SEQ ID NO: 1), S43P (hereinafter “design 21”, SEQ ID NO: 92), Y55P (hereinafter “design 22”, SEQ ID NO: 94), and S43P/Y55P (hereinafter “design 23”, SEQ ID NO: 96), were transfected together with the wild-type h492.1 heavy chain (SEQ ID NO: 2). On the indicated days after transfection, two microliters from each culture supernatant was spotted onto nitrocellulose in the pattern displayed on the lower left of the image, and the dot blots were developed using an anti-human Fc HRP-conjugated antibody.

FIG. 3 is a bar graph showing QSOX1 inhibition assays conducted by using 5,5-dithio-bis-2-nitrobenzoic acid (DTNB) to quantify the remaining dithiothreitol (DTT) after incubation with purified recombinant QSOX1 and h492.1 variants in HEK293F culture supernatants. Numbers on the x-axis are design numbers. Blue and gray bars are biological replicates, each of which were measured in triplicate. Error bars are standard deviations. The wild-type chimera h492.1 (wt) and the three light-chain mutants S43P (design 21), Y55P (design 22), and S43P/Y55P (design 23) were also assayed. HEK293F culture supernatants (25 μl) were mixed in a clear, flat-bottom, 96-well plate with 12.5 μl 40 nM QSOX1, and reactions were initiated by injection of 12.5 μl 600 μM DTT (final concentrations 10 nM QSOX1 and 150 μM DTT). Reactions were stopped after 30 min by adding 150 μl 500 μM DTNB, and absorbance at 412 nm was measured after 5 min in a Tecan microplate reader.

### DESCRIPTION OF SPECIFIC EMBODIMENTS OF THE INVENTION

The present invention, in some embodiments thereof, relates to chimeric Quiescin Sulfhydryl Oxidase (QSOX1) antibodies and uses of same.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Inhibition of extracellular human QSOX1 by a monoclonal antibody MAb 492.1 has been previously suggested to decrease tumor cell migration. Whilst humanizing Mab492.1, the h492.1 chimeric antibody was obtained by fusing the variable domains from the high-affinity (K_{D} approximately 1 nM) QSOX1-inhibiting murine antibody 492.1 onto a human IgG scaffold. Following this fusion, h492.1 could not be expressed to detectable levels in a recombinant cultured human cell system, restricting its further development. An automated computational approach was applied to improve this parameter by improving core packing at the vL-vH interface.

The present inventors identified a mutational space in the frameworks that can be harnessed towards improving expression whilst at least maintaining the QSOX1 inhibitory activity of the parent MAb 492.1.

Thus, according to an aspect of the invention there is provided an isolated antibody comprising an amino acid sequence of a light chain (vL) as set forth in SEQ ID NO: 1 and a heavy chain (vH) as set forth in SEQ ID NO: 2, wherein at least one of said vL and said vH comprises at least one amino acid substitution (single letter code) selected from the group consisting of:

| | |
|---|---|
| 34L | AS |
| 43L | SAPT |
| 46L | LV |
| 55L | YHPV |
| 89L | QH |
| 96L | LF |
| 37H | VF |
| 44H | GAKR |
| 45H | LF |
| 58H | DE |
| 94H | FY |
| 104H | SA |
| 105H | FLY |
| 106H | AENPQ |

wherein said antibody binds QSOX1.

Antibodies of some embodiments of the invention have improved Rosetta energy relative to an antibody comprising SEQ ID NO:1 and SEQ ID NO: 2. The Rosetta energy is a compound function of van der Waals packing, hydrogen bonding, electorsatics, solvation and statistical terms inferred from molecular structures of proteins.

As used herein, the term “QSOX1” relates to the Quiescin Sulfhydryl Oxidase 1. The protein accession number for the long variant of human QSOX1 on the NCBI database is NP_002817, and the accession number for the short form of human QSOX1 is NP_001004128.

As mentioned, the antibody binds QSOX1.

According to a specific embodiment, binding is determined by a Western blot assay, specifically, a dot-blot assay, as described in the Examples section which follows.

According to a specific embodiment, the binding of the antibody to QSOX1 is at least about the same affinity as that of MAb492.1 (as described in WO2013/132495), or h492.1 (the chimeric antibody), having a vH as set forth in SEQ ID NO: 2 and a vL as set forth in SEQ ID NO: 1).

Affinity should be determined in the same assay under the same assay conditions. The skilled artisan would know which assay to select. Examples include, but are not limited to surface plasmon resonance (SPR) or ELISA. As a general comment when comparing biological features, such are preferably determined using the assay under the same assay conditions.

According to a specific embodiment, the antibody is a recombinant antibody, produced using recombinant DNA technology, as further described hereinbelow.

According to a specific embodiment, the antibody is of the IgG1 serotype.

According to a specific embodiment, the at least one amino acid substitution is one amino acid substitution.

According to a specific embodiment, the at least one amino acid substitution comprises at least two amino acid substitutions.

According to a specific embodiment, the at least one amino acid substitution comprises at least three amino acid substitutions.

According to a specific embodiment, the at least one amino acid substitution comprises at least four amino acid substitutions.

According to a specific embodiment, the at least one amino acid substitution comprises at least five amino acid substitutions.

According to a specific embodiment, the at least one amino acid substitution comprises at least six amino acid substitutions.

According to a specific embodiment, the at least one amino acid substitution comprises at least seven amino acid substitutions.

According to a specific embodiment, the at least one amino acid substitution comprises at least eight amino acid substitutions.

According to a specific embodiment, the at least one amino acid substitution comprises at least nine amino acid substitutions.

According to a specific embodiment, the at least one amino acid substitution comprises at least 10 amino acid substitutions.

According to a specific embodiment, the at least one amino acid substitution comprises two amino acid substitutions.

According to a specific embodiment, the at least one amino acid substitution comprises three amino acid substitutions.

According to a specific embodiment, the at least one amino acid substitution comprises four amino acid substitutions.

According to a specific embodiment, the at least one amino acid substitution comprises five amino acid substitutions.

According to a specific embodiment, the at least one amino acid substitution comprises six amino acid substitutions.

According to a specific embodiment, the at least one amino acid substitution comprises seven amino acid substitutions.

According to a specific embodiment, the at least one amino acid substitution comprises eight amino acid substitutions.

According to a specific embodiment, the at least one amino acid substitution comprises nine amino acid substitutions.

According to a specific embodiment, the at least one amino acid substitution comprises two amino acid substitutions.

According to a specific embodiment, the at least one amino acid substitution comprises 10 amino acid substitutions.

According to a specific embodiment, the at least one amino acid substitution comprises 2-20 amino acid substitutions.

According to a specific embodiment, the at least one amino acid substitution comprises 4-20 amino acid substitutions.

According to a specific embodiment, the at least one amino acid substitution comprises 2-10 amino acid substitutions.

According to a specific embodiment, the at least one amino acid substitution comprises 5-10 amino acid substitutions.

As used, herein “amino acid substitution” or “amino acid replacement” refers to a change from one amino acid to a different amino acid.

For recombinant production the replacing amino acid is naturally occurring.

For other means of production, or for further modification, the amino acid can be non-naturally occurring homolog of the amino acids in the mutational space as defined above.

According to a specific embodiment, all the amino acid substitutions are at the vL.

According to a specific embodiment, all the amino acid substitutions are at the vH.

According to a specific embodiment, all the amino acid substitutions are at both the vL and vH.

According to a specific embodiment, the at least one amino acid substitution is at said position 43L.

According to a specific embodiment, the at least one amino acid substitution is at said position 55L.

According to a specific embodiment, the antibody is set forth in SEQ ID NOs: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80 and 82 (having the matching vH and vL chains), also designated as Designs 1-20.

According to a specific embodiment, the antibody exhibits an increased expression yield when expressed in HEK293F cells as determined by Western blot and at least about the same QSOX1 inhibitory activity as determined by sulfhydryl oxidase assay as that of an antibody designated chimeric h492.1 comprising an amino acid sequence of vL as set forth in SEQ ID NO: 1 and a vH as set forth in SEQ ID NO: 2.

Both assays are described in the Examples section, which follows.

As used herein “increased” refers to at least about 5%, 10%, 20%, 30%, 40%, 50%, 70%, 80%, 90%, 1.5 fold, 2 fold, 3 fold, 5 fold, 10 fold, 20 fold, 50 fold or even 100 fold increase.

According to a specific embodiment, the antibody is set forth in SEQ ID NOs: 8, 48 and 38, 78 (DES 3 and DES 18, respectively).

According to a specific embodiment, the antibody is set forth in SEQ ID NOs: 8, 48 (DES 3).

According to a specific embodiment, the antibody is set forth in SEQ ID NOs: 38, 78 (DES 18).

According to a specific embodiment, the antibody comprises the amino acid sequences of Designs 21, 22 or 23.

According to a specific embodiment, the antibody inhibits QSOX1 activity in mediating laminin incorporation in the basement membrane.

According to a specific embodiment, the activity is assayed by at least one of an immunofluorescence (IF) staining assay of an extracellular matrix or a Western blot assay detecting for soluble laminin.

As used herein, the term “laminin” refers to a human laminin protein. Typically, laminins are trimeric proteins that contain an α-chain, a β-chain, and a γ-chain (found in five, four, and three genetic variants, respectively). Thus, the term laminin as used herein encompasses any type of human laminin, including any of the different chain combinations or any individual subunits of laminin. The different chains and trimer molecules differ with respect to their tissue distribution apparently reflecting diverse functions in vivo. Exemplary laminin subunits of the present invention include, but are not limited to, LAMA1, LAMA2, LAMA3, LAMA4, LAMA5, LAMB1, LAMB2, LAMB3, LAMB4, LAMC1, LAMC2 and LAMC3.

According to an embodiment of the present invention, the laminin comprises an alpha 4 chain.

According to a specific embodiment, the laminin is laminin-411 or laminin-421.

The term “laminin assembly” refers to the incorporation of laminin proteins into the basal lamina (i.e. one of the layers of the basement membrane). Typically, laminin is secreted from cells (e.g., fibroblasts, epithelial cells, tumor cells) and is incorporated into cell-associated extracellular matrices where they form independent networks and are associated with type IV collagen networks via entactin, fibronectin and perlecan.

The term “basement membrane” or “laminin-comprising basement membrane” refers to the thin layer of fibers, which anchors and supports the epithelium and endothelium and comprises the basal lamina (i.e. comprising laminin).

The phrase “inhibiting or preventing laminin assembly” refers to reducing, reversing, attenuating, minimizing, suppressing or halting laminin assembly in a basement membrane. According to one embodiment, inhibiting or preventing laminin assembly is by at least about 10%, by at least about 20%, by at least about 30%, by at least about 40%, by at least about 50%, by at least about 60%, by at least about 70%, by at least about 80%, by at least about 90% or by about 100%, as compared to laminin assembly in the absence of the anti-QSOX1 antibody (as described hereinabove). Thus, according to an embodiment of the invention laminin is not incorporated into the basement membrane.

Laminin, which is not incorporated into the basal membrane, can be found in soluble form (e.g., in the culture medium of in vitro cultured cells). Thus, monitoring reduction in laminin assembly can be monitored by e.g., immunofluorescence (IF) staining of the extracellular matrix or by Western blotting of the soluble laminin (i.e. that which was not incorporated into the basal membrane).

According to an embodiment of the invention, the activity of the antibody in inhibiting QSOX1 activity is assayed by at least one of an immunofluorescence (IF) staining assay of the extracellular matrix or Western blot assay for soluble laminin (i.e. that which is not incorporated into the basal membrane).

The term “antibody” as used in this invention includes intact molecules that are capable of specifically binding to an epitope of an antigen, in this case QSOX1.

The term “isolated” refers to at least partially separated from the natural environment e.g., from a cell expressing same.

As used herein, the terms “complementarity-determining region” or “CDR” are used interchangeably to refer to the antigen binding regions found within the variable region of the heavy and light chain polypeptides. Generally, antibodies comprise three CDRs in each of the VH (CDR HI or HI; CDR H2 or H2; and CDR H3 or H3) and three in each of the VL (CDR LI or LI; CDR L2 or L2; and CDR L3 or L3). In this case, the amino acid sequences of the CDRs are as set forth in SEQ ID NOs: 83-88.

Also provided are polynucleotides encoding for the antibodies of some embodiments of the invention. Such polynucleotides can be used for recombinant expression typically in cell cultures as further described hereinbelow (e.g., HEK293F cells grown in suspension ThermoFisher; www(dot) thermofisher(dot)com/order/catalog/product/R79007) or host animals.

Exemplary polynucleotides include, but are not limited to SEQ ID NOs: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79 and 81.

In the context of an antibody comprising CDR sequences which are not completely shared by 492.1 (WO2013/132495), the present teachings relate to an antibody is a broader manner, such as including antibody fragments.

Thus, according to an aspect of the invention there is provided an antibody comprising an antigen recognition domain comprising complementarity-determining regions as set forth in GFSLTGYG (CDRHI) (SEQ ID NO: 83), IWGDGRT (CDRH2) (SEQ ID NO: 84) and ASDYYGSGX₁X₂X₃Y, (CDRH3) (SEQ ID NO: 97), QDVSTA (CDRLI) (SEQ ID NO: 86), SAS (CDRL2) (SEQ ID NO: 87) and QQHYSIPX₄T (CDRL3) (SEQ ID NO: 98), wherein X₁, X₂, X₃ and X₄ is any amino acid, wherein said antibody binds QSOX1, wherein when the antibody comprises CDRH3 set forth in SEQ ID NO: 85 said CDRL3 is not as set forth in SEQ ID NO: 88.

According to an embodiment of the invention, the X₁ is selected from the group consisting of S and A.

According to an embodiment of the invention, the X₂ is selected from the group consisting of F, L and Y.

According to an embodiment of the invention, the X₃ is selected from the group consisting of A, E, N, P and Q.

According to an embodiment of the invention, the X₄ is F (SEQ ID NO: 119).

According to an embodiment of the invention, the CDRH3 is selected from the group consisting of ASDYYGSGALEY (SEQ ID NO: 99), ASDYYGSGALAY (SEQ ID NO: 100), ASDYYGSGAFEY (SEQ ID NO: 101), ASDYYGSGALEY (SEQ ID NO: 102), ASDYYGSGSFPY (SEQ ID NO: 103), ASDYYGSGSFNY (SEQ ID NO: 104), ASDYYGSGALEY (SEQ ID NO: 105), ASDYYGSGSLEY (SEQ ID NO: 106), ASDYYGSGSYQY (SEQ ID NO: 107), ASDYYGSGALQY (SEQ ID NO: 108), ASDYYGSGSLAY (SEQ ID NO: 109), ASDYYGSGAFQY (SEQ ID NO: 110), ASDYYGSGSFQY (SEQ ID NO: 111), ASDYYGSGSLAY (SEQ ID NO: 112), ASDYYGSGAYEY (SEQ ID NO: 113), ASDYYGSGSYQY (SEQ ID NO: 114), ASDYYGSGSYQY (SEQ ID NO: 115), ASDYYGSGALAY (SEQ ID NO: 116), ASDYYGSGALAY (SEQ ID NO: 117) and ASDYYGSGSYAY (SEQ ID NO: 118).

It will be appreciated that conservative substitutions are contemplated herein. Conservative substitution tables providing functionally similar amino acids are well known in the art. Guidance concerning which amino acid changes are likely to be phenotypically silent can also be found in Bowie et al., 1990, Science 247: 1306 1310. Such conservatively modified variants are in addition to and do not exclude polymorphic variants, interspecies homologs, and alleles. Typical conservative substitutions include but are not limited to: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)). Amino acids can be substituted based upon properties associated with side chains, for example, amino acids with polar side chains may be substituted, for example, Serine (S) and Threonine (T); amino acids based on the electrical charge of a side chains, for example, Arginine (R) and Histidine (H); and amino acids that have hydrophobic side chains, for example, Valine (V) and Leucine (L). As indicated, changes are typically of a minor nature, such as conservative amino acid substitutions that do not significantly affect the folding or activity of the protein.

The term “antibody” as used in this invention includes intact molecules as well as functional fragments thereof, such as Fab, F(ab′)2, and Fv that are capable of binding to macrophages. These functional antibody fragments are defined as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)2, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; and (5) Single chain antibody (“SCA”), a genetically engineered molecule containing the variable region of the light chain and the variable region of the heavy chain, linked by a suitable polypeptide linker as a genetically fused single chain molecule.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference). QSOX1 peptides used for immunization may comprise between 50-100 amino acids, between 50-150 amino acids, between 50-200 amino acids, between 50-232 amino acids, between 100-200 amino acids or between 150-232 amino acids.

Antibody fragments according to some embodiments of the invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains (e.g. [Gly₄Ser]₃ as taught in Example 2, hereinbelow). Methods for producing sFvs are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

Humanized forms of non-human (e.g., murine) antibodies are chimeric molecules of immunoglobulins, immunoglobulin chains or fragments thereof (such as Fv, Fab, Fab′, F(ab′).sub.2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source, which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

Nucleic acid sequences encoding for the polypeptides of some embodiments of the invention may be optimized for expression in a desired cell system. Examples of such sequence modifications include, but are not limited to, an altered G/C content to more closely approach that typically found in the species of interest, and the removal of codons atypically found in the species commonly referred to as codon optimization.

The phrase “codon optimization” refers to the selection of appropriate DNA nucleotides for use within a structural gene or fragment thereof that approaches codon usage within the species of interest.

Also provided is a cell, comprising the polynucleotide of some embodiments of the invention. It will be appreciated that both polynucleotides (vH and vL) can be expressed from the same cell or from different cells (e.g., different cell cultures) after which they are allowed to fold together.

Typically is order to express a coding sequence of interest, the polynucleotide is ligated into a nucleic acid expression construct suitable for cell expression. Such a nucleic acid construct includes a promoter sequence for directing transcription of the polynucleotide sequence in the cell in a constitutive or inducible manner.

Hence, according to specific embodiments, there is provided nucleic acid construct comprising the polynucleotide and a regulatory element for directing expression of said polynucleotide in a host cell.

According to specific embodiments, the regulatory element is a heterologous regulatory element.

The nucleic acid construct (also referred to herein as an “expression vector”) of some embodiments of the invention includes additional sequences which render this vector suitable for replication and integration in prokaryotes, eukaryotes, or preferably both (e.g., shuttle vectors). In addition, a typical cloning vector may also contain a transcription and translation initiation sequence, transcription and translation terminator and a polyadenylation signal. By way of example, such constructs will typically include a 5′ LTR, a tRNA binding site, a packaging signal, an origin of second-strand DNA synthesis, and a 3′ LTR or a portion thereof.

The nucleic acid construct of some embodiments of the invention typically includes a signal sequence for secretion of the antibody from a host cell in which it is placed.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for some embodiments of the invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

Polyadenylation sequences can also be added to the expression vector in order to increase the efficiency of vH and vL mRNA translation. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for some embodiments of the invention include those derived from SV40.

In addition to the elements already described, the expression vector of some embodiments of the invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

The expression vector of some embodiments of the invention can further include additional polynucleotide sequences that allow, for example, the translation of several proteins from a single mRNA such as an internal ribosome entry site (IRES) and sequences for genomic integration of the promoter-chimeric polypeptide.

Examples for mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

According to a specific embodiment, the vector is as described in Tiller et al. Journal of Immunological Methods 329 (2008) 112-124. Also contemplated is the use of an HCMV promoter.

Expression vectors containing regulatory elements from eukaryotic viruses such as retroviruses can be also used. SV40 vectors include pSVT7 and pMT2. Vectors derived from bovine papilloma virus include pBV-1MTHA, and vectors derived from Epstein Bar virus include pHEBO, and p2O5. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

Various methods can be used to introduce the expression vector of some embodiments of the invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

Introduction of nucleic acids by viral infection offers several advantages over other methods such as lipofection and electroporation, since higher transfection efficiency can be obtained due to the infectious nature of viruses.

Examples of eukaryotic cells which may be used along with the teachings of the invention include but are not limited to, mammalian cells, fungal cells, yeast cells, insect cells, algal cells or plant cells.

According to specific embodiments, the cell is a mammalian cell.

According to specific embodiment, the cell is a human cell.

According to a specific embodiment, the cell is a cell line.

According to another specific embodiment, the cell is a primary cell.

The cell may be derived from a suitable tissue including but not limited to blood, muscle, nerve, brain, heart, lung, liver, pancreas, spleen, thymus, esophagus, stomach, intestine, kidney, testis, ovary, hair, skin, bone, breast, uterus, bladder, spinal cord, or various kinds of body fluids. The cells may be derived from any developmental stage including embryo, fetal and adult stages, as well as developmental origin i.e., ectodermal, mesodermal, and endodermal origin.

Non limiting examples of mammalian cells include human embryonic kidney line (HEK293) Expi293 cells, or HEK293 cells subcloned for growth in suspension culture, Graham et al., J. Gen Virol., 36:59 1977); monkey kidney CV1 line transformed by SV40 (COS, e.g. COS-7, ATCC CRL 1651); baby hamster kidney cells (BHK, ATCC CCL 10); mouse sertoli cells (TM4, Mather, Biol. Reprod., 23:243-251 1980); monkey kidney cells (CV1 ATCC CCL 70); African green monkey kidney cells (VERO-76, ATCC CRL-1587); human cervical carcinoma cells (HeLa, ATCC CCL 2); NIH3T3, Jurkat, canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci., 383:44-68 1982); MRC 5 cells; FS4 cells; and a human hepatoma line (Hep G2), PER.C6, K562, and Chinese hamster ovary cells (CHO).

According to some embodiments of the invention, the mammalian cell is selected from the group consisting of a Chinese Hamster Ovary (CHO), HEK293, PER.C6, HT1080, NS0, Sp2/0, BHK, Namalwa, COS, HeLa and Vero cell.

According to some embodiments of the invention, the mammalian cell is HEK293.

According to another aspect of the present invention, there is provided a method of producing an antibody to QSOX1, the method comprising:

(a) culturing cells, which comprise polynucleotide(s) of the invention under conditions, which allow for expression of the vH and/or vL chains;
(b) recovering the vH and/or vL chains from the cells.

Transformed cells are cultured under effective conditions, which allow for the expression of high amounts of recombinant polypeptide. Effective culture conditions include, but are not limited to, effective media, bioreactor, temperature, pH and oxygen conditions that permit protein production. An effective medium refers to any medium in which a cell is cultured to produce the recombinant polypeptide of the present invention. Such a medium typically includes an aqueous solution having assimilable carbon, nitrogen and phosphate sources, and appropriate salts, minerals, metals and other nutrients, such as vitamins.

Cells of the present invention can be cultured in conventional fermentation bioreactors, shake flasks, test tubes, microtiter dishes and petri plates. Culturing can be carried out at a temperature, pH and oxygen content appropriate for a recombinant cell. Such culturing conditions are within the expertise of one of ordinary skill in the art.

Depending on the vector and host system used for production, resultant polypeptides of the present invention may either remain within the recombinant cell, secreted into the fermentation medium, secreted into a space between two cellular membranes; or retained on the outer surface of a cell or viral membrane.

Following a predetermined time in culture, recovery of the recombinant polypeptide is effected.

If the protein is expressed in the cell, the cell membrane is preferably disrupted so as to release the polypeptide.

Cell disruption may be effected using methods known in the art including homogenization. Further steps of purification are provided below.

When released to the medium, the phrase “recovering the recombinant polypeptide” refers to collecting the whole fermentation medium containing the polypeptide and need not imply additional steps of separation or purification.

Notwithstanding the above, polypeptides of some embodiments of the invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin. A chromatography, protein A/G/L separation, mix mode chromatography, metal affinity chromatography, Lectins affinity chromatography, chromatofocusing and differential solubilization.

According to a specific embodiment, following purification the antibody is purified to a level of at least 95%, 96%, 97%, 98%, 99% or even higher level of purity (less than 5% host cell contaminants w/w or w/v).

According to specific embodiments, following synthesis and purification, the therapeutic efficacy of the antibody can be assayed such as by determining binding to QSOX1, activity by sulfhydryl oxidase assay, expression by Western blotting etc. or in vivo in appropriate animal models such as for cancer and fibrosis.

It will be appreciated that inhibiting or preventing laminin assembly may be advantageous in situations in which excess connective tissue is produced in a non-structured manner in an organ or tissue in a reparative or reactive process, such as fibrosis. Thus, while further reducing the present invention to practice, inhibition of QSOX1 and subsequently generation of soluble laminin may be therapeutic for fibrotic processes.

It will be appreciated that laminins are an important biologically active part of the basal lamina and basal membrane influencing cell adhesion, signaling, migration, phenotype, differentiation and survival. An exemplary cell migration of the present invention comprises tumor cell migration leading to metastasis.

Accordingly, inhibiting or preventing laminin assembly may be advantageous in situations in which inhibition of cell migration is warranted. The cell may comprise, for example, a brain cell, a neuron, a cardiac cell, a muscle cell, a skin cell, a bone cell, a pancreatic cell, a liver cell, a kidney cell, an intestinal cell, a spleen cell, a respiratory cell, a lung cell, a lymphocyte or a monocyte. The cell of the present invention may comprise a healthy cell or may alternately comprise a mutated cell (e.g., a tumor cell).

According to one embodiment, inhibiting or preventing cell migration refers to reducing, reversing, attenuating, minimizing, suppressing or halting migration of a cell (e.g., tumor cell) via a laminin-comprising basement membrane.

According to one embodiment, inhibiting or preventing cell migration is by at least about 10%, by at least about 20%, by at least about 30%, by at least about 40%, by at least about 50%, by at least about 60%, by at least about 70%, by at least about 80%, by at least about 90% or by at least about 100%, as compared to cell migration via a laminin-comprising basement membrane in the absence of the antibody (as described hereinabove).

The methods of the present invention (e.g., inhibiting cell migration) may be effected in vitro, in vivo or ex vivo.

As mentioned, the ability to modulate cell migration can be used as a therapeutic modality.

Accordingly, one specific use for the antibodies of the present invention is for preventing or treating a laminin-associated disease or condition in a subject in need thereof.

The phrase “preventing or treating” refers to inhibiting or arresting the development of a disease, disorder or condition and/or causing the reduction, remission, or regression of a disease, disorder or condition or keeping a disease, disorder or medical condition from occurring in a subject who may be at risk for the disease disorder or condition, but has not yet been diagnosed as having the disease disorder or condition. Those of skill in the art will understand that various methodologies and assays can be used to assess the development of a disease, disorder or condition, and similarly, various methodologies and assays may be used to assess the reduction, remission or regression of a disease, disorder or condition.

As used herein, the term “subject” refers to an animal, preferably a mammal, most preferably a human being, including both young and old human beings of both genders who suffer from or are predisposed to a laminin-associated disease or condition.

As used herein, the term “laminin-associated disease or condition” refers to a disease or condition in which laminin function is associated with the onset or progression of a disease.

According to one embodiment, the laminin-associated disease or condition is a tumor.

Examples of tumors include, but are not limited to, carcinoma, blastoma and sarcoma. Particular examples of cancerous diseases but are not limited to: myeloproliferative diseases, such as solid tumors benign meningioma, mixed tumors of salivary gland, colonic adenomas; adenocarcinomas, such as small cell lung cancer, kidney, uterus, prostate, bladder, ovary, colon, sarcomas, liposarcoma, myxoid, synovial sarcoma, rhabdomyosarcoma (alveolar), extraskeletal myxoid chonodrosarcoma, Ewing's tumor; other include testicular and ovarian dysgerminoma, retinoblastoma, Wilms' tumor, neuroblastoma, malignant melanoma, mesothelioma, breast, skin, prostate, and ovarian.

According to an embodiment, the tumor is a metastasizing solid tumor (e.g., formed by metastatic cancer cells).

According to an embodiment, the tumor is an adenocarcinoma.

According to one embodiment the tumor is a cancer.

Types of cancerous diseases amenable to treatment by the methods of some embodiments of the invention include benign tumors, warts, polyps, pre-cancers, and malignant tumors/cancers.

Specific examples of cancerous diseases which can be treated using the methods of the present invention include, but are not limited to, tumors of the gastrointestinal tract (colon carcinoma, rectal carcinoma, colorectal carcinoma, colorectal cancer, colorectal adenoma, hereditary nonpolyposis type 1, hereditary nonpolyposis type 2, hereditary nonpolyposis type 3, hereditary nonpolyposis type 6; colorectal cancer, hereditary nonpolyposis type 7, small and/or large bowel carcinoma, esophageal carcinoma, tylosis with esophageal cancer, stomach carcinoma, pancreatic carcinoma, pancreatic endocrine tumors), endometrial carcinoma, dermatofibrosarcoma protuberans, gallbladder carcinoma, Biliary tract tumors, prostate cancer, prostate adenocarcinoma, renal cancer (e.g., Wilms' tumor type 2 or type 1), liver cancer (e.g., hepatoblastoma, hepatocellular carcinoma, hepatocellular cancer), bladder cancer, embryonal rhabdomyosarcoma, germ cell tumor, trophoblastic tumor, testicular germ cells tumor, immature teratoma of ovary, uterine, epithelial ovarian, sacrococcygeal tumor, choriocarcinoma, placental site trophoblastic tumor, epithelial adult tumor, ovarian carcinoma, serous ovarian cancer, ovarian sex cord tumors, cervical carcinoma, uterine cervix carcinoma, small-cell and non-small cell lung carcinoma, nasopharyngeal, breast carcinoma (e.g., ductal breast cancer, invasive intraductal breast cancer, sporadic; breast cancer, susceptibility to breast cancer, type 4 breast cancer, breast cancer-1, breast cancer-3; breast-ovarian cancer), squamous cell carcinoma (e.g., in head and neck), neurogenic tumor, astrocytoma, ganglioblastoma, neuroblastoma, lymphomas (e.g., Hodgkin's disease, non-Hodgkin's lymphoma, B cell, Burkitt, cutaneous T cell, histiocytic, lymphoblastic, T cell, thymic), gliomas, adenocarcinoma, adrenal tumor, hereditary adrenocortical carcinoma, brain malignancy (tumor), various other carcinomas (e.g., bronchogenic large cell, ductal, Ehrlich-Lettre ascites, epidermoid, large cell, Lewis lung, medullary, mucoepidermoid, oat cell, small cell, spindle cell, spinocellular, transitional cell, undifferentiated, carcinosarcoma, choriocarcinoma, cystadenocarcinoma), ependimoblastoma, epithelioma, erythroleukemia (e.g., Friend, lymphoblast), fibrosarcoma, giant cell tumor, glial tumor, glioblastoma (e.g., multiforme, astrocytoma), glioma hepatoma, heterohybridoma, heteromyeloma, histiocytoma, hybridoma (e.g., B cell), hypernephroma, insulinoma, islet tumor, keratoma, leiomyoblastoma, leiomyosarcoma, leukemia (e.g., acute lymphatic, acute lymphoblastic, acute lymphoblastic pre-B cell, acute lymphoblastic T cell leukemia, acute-megakaryoblastic, monocytic, acute myelogenous, acute myeloid, acute myeloid with eosinophilia, B cell, basophilic, chronic myeloid, chronic, B cell, eosinophilic, Friend, granulocytic or myelocytic, hairy cell, lymphocytic, megakaryoblastic, monocytic, monocytic-macrophage, myeloblastic, myeloid, myelomonocytic, plasma cell, pre-B cell, promyelocytic, subacute, T cell, lymphoid neoplasm, predisposition to myeloid malignancy, acute nonlymphocytic leukemia), lymphosarcoma, melanoma, mammary tumor, mastocytoma, medulloblastoma, mesothelioma, metastatic tumor, monocyte tumor, multiple myeloma, myelodysplastic syndrome, myeloma, nephroblastoma, nervous tissue glial tumor, nervous tissue neuronal tumor, neurinoma, neuroblastoma, oligodendroglioma, osteochondroma, osteomyeloma, osteosarcoma (e.g., Ewing's), papilloma, transitional cell, pheochromocytoma, pituitary tumor (invasive), plasmacytoma, retinoblastoma, rhabdomyosarcoma, sarcoma (e.g., Ewing's, histiocytic cell, Jensen, osteogenic, reticulum cell), schwannoma, subcutaneous tumor, teratocarcinoma (e.g., pluripotent), teratoma, testicular tumor, thymoma and trichoepithelioma, gastric cancer, fibrosarcoma, glioblastoma multiforme; multiple glomus tumors, Li-Fraumeni syndrome, liposarcoma, lynch cancer family syndrome II, male germ cell tumor, mast cell leukemia, medullary thyroid, multiple meningioma, endocrine neoplasia myxosarcoma, paraganglioma, familial nonchromaffin, pilomatricoma, papillary, familial and sporadic, rhabdoid predisposition syndrome, familial, rhabdoid tumors, soft tissue sarcoma, and Turcot syndrome with glioblastoma.

According to a specific embodiment of this aspect of the present invention, the cancers which may be treated in accordance with the present teachings, include but are not limited to, prostate cancer, lung cancer, breast cancer, cervical cancer, urachus cancer, vaginal cancer, colon cancer, esophagus cancer, pancreatic cancer, throat cancer, stomach cancer and myeloid leukemia.

According to one embodiment, the laminin-associated disease or condition is associated with fibrosis.

The term “fibrosis” refers to a formation or a presence of excess connective tissue in an organ or tissue. It may occur as a repair or replacement response to a stimulus such as tissue injury or inflammation.

Examples of disorders involving fibrosis include, but are not limited to, liver fibrosis, pulmonary fibrosis, renal fibrosis, pancreatic fibrosis, scleroderma, connective tissue diseases, scarring, skin fibrosis, cardiac fibrosis, organ transplant, vascular stenosis, restenosis, arterial fibrosis, arthrofibrosis, breast fibrosis, muscle fibrosis, retroperitoneal fibrosis, thyroid fibrosis, lymph node fibrosis, bladder fibrosis, pleural fibrosis and COPD.

According to one embodiment, the laminin-associated disease or condition is a bacterial disease, a viral disease or a parasitic disease.

An exemplary parasitic disease, which may be treated by the teachings of the present invention, includes African trypanosomiasis.

According to the present teachings, in order to treat the laminin-associated disease or condition, the subject is administered with the anti-QSOX1 antibody (or antibody fragment) of some embodiments of the invention, as further detailed hereinabove.

Each of the antibody or antibody fragments described hereinabove can be administered to the individual per se or as part of a pharmaceutical composition which also includes a physiologically acceptable carrier. The purpose of a pharmaceutical composition is to facilitate administration of the active ingredient to an organism.

As used, herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the anti-QS OX1 antibody or fragment thereof accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide).

Alternately, one may administer the pharmaceutical composition in a local rather than systemic manner, for example, via injection of the pharmaceutical composition directly into a tissue region of a patient.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations, which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents, which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (anti-QSOX1 antibody or fragment thereof) effective to prevent, alleviate or ameliorate symptoms of a disorder (e.g., laminin-associated disease or condition) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Animal models for laminin-associated diseases include, for example, the murine animal model for liver fibrosis [see e.g., review paper by Hiromitsu Hayashi and Takao Sakai1, Amer Journal Physiol—GI (2011) 300(5): G729-G738] and the murine animal model for metastatic breast cancer [Anna Fantozzi and Gerhard Christofori, Breast Cancer Research (2006) 8:212].

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide the active ingredient at a sufficient amount to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

According to one embodiment, the antibody of some embodiments of the invention is used in conjunction with another agent capable of treating a laminin-associated disease or condition in a subject (e.g. tumor). In such cases, the antibody may be administered to the subject prior to, concomitantly with, or following said other agent (e.g. within a time frame of 30 minutes, 1 hour, 2 hours, 4 hours, 6 hours, 12 hours, 24 hours, 36 hours, 48 hours, 3 days, 4 days, 5 days, 6 days, 7 days, 10 days, 14 days, 21 days, 30 days, 60 days, 90 days or 120 days of each other).

Exemplary agents include, but are not limited to, chemotherapeutic agents (e.g. cytotoxic drugs), hormonal therapeutic agents, radiotherapeutic agents, anti-proliferative agents, and combinations thereof.

Non-limiting examples of chemotherapeutic agents include, but are not limited to, platinum-based drugs (e.g., oxaliplatin, cisplatin, carboplatin, spiroplatin, iproplatin, satraplatin, etc.), alkylating agents (e.g., cyclophosphamide, ifosfamide, chlorambucil, busulfan, melphalan, mechlorethamine, uramustine, thiotepa, nitrosoureas, etc.), anti-metabolites (e.g., 5-fluorouracil, azathioprine, 6-mercaptopurine, methotrexate, leucovorin, capecitabine, cytarabine, floxuridine, fludarabine, gemcitabine (Gemzar®), pemetrexed (ALIMTA®), raltitrexed, etc.), plant alkaloids (e.g., vincristine, vinblastine, vinorelbine, vindesine, podophyllotoxin, paclitaxel (Taxol®), docetaxel (Taxotere®), etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), pharmaceutically acceptable salts thereof, stereoisomers thereof, derivatives thereof, analogs thereof, and combinations thereof.

According to a specific embodiment, the chemotherapeutic agent is Doxorubicin.

It will be appreciated that the antibody may allow lower doses of chemotherapeutic agents to be used (e.g. doses which are less than the current gold standard), thus minimizing adverse toxicity typically associated with the use of such treatments.

Examples of hormonal therapeutic agents include, but are not limited to, aromatase inhibitors (e.g., aminoglutethimide, anastrozole (Arimidex®), letrozole (Femora®), vorozole, exemestane (Aromasin®), 4-androstene-3,6,17-trione (6-OXO), 1,4,6-androstatrien-3,17-dione (ATD), formestane (Lentaron®), etc.), selective estrogen receptor modulators (e.g., bazedoxifene, clomifene, fulvestrant, lasofoxifene, raloxifene, tamoxifen, toremifene, etc.), steroids (e.g., dexamethasone), finasteride, and gonadotropin-releasing hormone agonists (GnRH) such as goserelin, pharmaceutically acceptable salts thereof, stereoisomers thereof, derivatives thereof, analogs thereof, and combinations thereof.

Radiation therapy includes, but is not limited to, fractionated radiotherapy, non-fractionated radiotherapy and hyper-fractionated radiotherapy, and combination radiation and chemotherapy. Types of radiation also include ionizing (gamma) radiation, particle radiation, low energy transmission (LET), high energy transmission (HET), ultraviolet radiation, infrared radiation, visible light, and photosensitizing radiation.

Exemplary anti-proliferative agents include mTOR inhibitors such as sirolimus (rapamycin), temsirolimus (CCI-779), and everolimus (RAD001); Akt inhibitors such as IL6-hydroxymethyl-chiro-inositol-2-(R)-2-O-methyl-3-O-octadecyl-sn-glycerocarbonate, 9-methoxy-2-methylellipticinium acetate, 1,3-dihydro-1-(1-((4-(6-phenyl-1H-imidazo [4,5-g]quinoxalin-7-yl)phenyl)me-thyl)-4-piperidinyl)-2H-benzimidazol-2-one, 10-(4′-(N-diethylamino)butyl)-2-chlorophenoxazine, 3-formylchromone thiosemicarbazone (Cu(II)Cl.sub.2 complex), API-2, a 15-mer peptide derived from amino acids 10-24 of the proto-oncogene TCL1 (Hiromura et al., J. Biol. Chem., 279:53407-53418 (2004), KP372-1, and the compounds described in Kozikowski et al., J. Am. Chem. Soc., 125:1144-1145 (2003) and Kau et al., Cancer Cell, 4:463-476 (2003); and combinations thereof.

It will be appreciated that the antibody of some embodiments of the present invention may be attached to a detectable moiety in order to enable detection (e.g., in vivo detection) of the antibody.

Various types of detectable or reporter moieties may be conjugated to the antibody of the invention. These include, but not are limited to, a radioactive isotope (such as ^{[125]}iodine), a phosphorescent chemical, a chemiluminescent chemical, a fluorescent chemical (fluorophore), an enzyme, a fluorescent polypeptide, an affinity tag, and molecules (contrast agents) detectable by Positron Emission Tomography (PET) or Magnetic Resonance Imaging (MRI).

Examples of suitable fluorophores include, but are not limited to, phycoerythrin (PE), fluorescein isothiocyanate (FITC), Cy-chrome, rhodamine, green fluorescent protein (GFP), blue fluorescent protein (BFP), Texas red, PE-Cy5, and the like. For additional guidance regarding fluorophore selection, methods of linking fluorophores to various types of molecules see Richard P. Haugland, “Molecular Probes: Handbook of Fluorescent Probes and Research Chemicals 1992-1994”, 5th ed., Molecular Probes, Inc. (1994); U.S. Pat. No. 6,037,137 to Oncoimmunin Inc.; Hermanson, “Bioconjugate Techniques”, Academic Press New York, N.Y. (1995); Kay M. et al., 1995. Biochemistry 34:293; Stubbs et al., 1996. Biochemistry 35:937; Gakamsky D. et al., “Evaluating Receptor Stoichiometry by Fluorescence Resonance Energy Transfer,” in “Receptors: A Practical Approach,” 2nd ed., Stanford C. and Horton R. (eds.), Oxford University Press, UK. (2001); U.S. Pat. No. 6,350,466 to Targesome, Inc.]. Fluorescence detection methods which can be used to detect the antibody when conjugated to a fluorescent detectable moiety include, for example, fluorescence activated flow cytometry (FACS), immunofluorescence confocal microscopy, fluorescence in-situ hybridization (FISH) and fluorescence resonance energy transfer (FRET).

Numerous types of enzymes may be attached to the antibody of the invention [e.g., horseradish peroxidase (HPR), beta-galactosidase, and alkaline phosphatase (AP)] and detection of enzyme-conjugated antibodies can be performed using ELISA (e.g., in solution), enzyme-linked immunohistochemical assay (e.g., in a fixed tissue), enzyme-linked chemiluminescence assay (e.g., in an electrophoretically separated protein mixture) or other methods known in the art [see e.g., Khatkhatay M I. and Desai M., 1999. J Immunoassay 20:151-83; Wisdom G B., 1994. Methods Mol Biol. 32:433-40; Ishikawa E. et al., 1983. J Immunoassay 4:209-327; Oellerich M., 1980. J Clin Chem Clin Biochem. 18:197-208; Schuurs A H. and van Weemen B K., 1980. J Immunoassay 1:229-49).

The affinity tag (or a member of a binding pair) can be an antigen identifiable by a corresponding antibody [e.g., digoxigenin (DIG) which is identified by an anti-DIG antibody) or a molecule having a high affinity towards the tag [e.g., streptavidin and biotin]. The antibody or the molecule which binds the affinity tag can be fluorescently labeled or conjugated to enzyme as described above.

Various methods, widely practiced in the art, may be employed to attach a streptavidin or biotin molecule to the antibody of the invention. For example, a biotin molecule may be attached to the antibody of the invention via the recognition sequence of a biotin protein ligase (e.g., BirA) as described in the Examples section, which follows, and in Denkberg, G. et al., 2000. Eur. J. Immunol. 30:3522-3532. Alternatively, a streptavidin molecule may be attached to an antibody fragment, such as a single chain Fv, essentially as described in Cloutier S M. et al., 2000. Molecular Immunology 37:1067-1077; Dubel S. et al., 1995. J Immunol Methods 178:201; Huston J S. et al., 1991. Methods in Enzymology 203:46; Kipriyanov S M. et al., 1995. Hum Antibodies Hybridomas 6:93; Kipriyanov S M. et al., 1996. Protein Engineering 9:203; Pearce L A. et al., 1997. Biochem Molec Biol Intl 42:1179-1188).

Functional moieties, such as fluorophores, conjugated to streptavidin are commercially available from essentially all major suppliers of immunofluorescence flow cytometry reagents (for example, Pharmingen or Becton-Dickinson).

According to some embodiments of the invention, biotin conjugated antibodies are bound to a streptavidin molecule to form a multivalent composition (e.g., a dimer or tetramer form of the antibody).

According to one embodiment, the antibody of some embodiments of the invention can be used for in vitro or ex vivo applications (e.g., for detection of QSOX1 levels in biological samples).

According to one embodiment, the antibody of the invention may be immobilized on a solid support (e.g., for formation of an immunocomplex between the antibody and QSOX1 proteins in ex vivo or in vitro settings). As used herein the phrase “solid support” refers to a non-aqueous matrix to which a reagent of interest (e.g., the antibody of this aspect of the present invention) can adhere. Examples of solid supports include, but are not limited to, solid supports formed partially or entirely of glass (e.g., controlled pore glass), polysaccharides (e.g., agarose), polyacrylamides, polystyrene, polyvinyl alcohol and silicones. In certain embodiments, depending on the context, the solid support can comprise the well of an assay plate; in others it is a purification column (e.g., an affinity chromatography column). This term also includes a discontinuous solid phase of discrete particles, such as those described in U.S. Pat. No. 4,275,149.

The agents described hereinabove may be included in a diagnostic kit/article of manufacture preferably along with appropriate instructions for use and labels indicating FDA approval for use in diagnosing and/or assessing efficiency of treatment of a laminin-associated disease.

According to another aspect of the present invention, there is provided a kit for detecting a level of QSOX1 in a biological sample.

Such a kit can include, for example, at least one container including at least one of the above described diagnostic agents (e.g., antibodies comprising an antigen recognition domain to QSOX1) and an imaging reagent packed in another container (e.g., enzymes, secondary antibodies, buffers, chromogenic substrates, fluorogenic material). The kit may also include appropriate buffers and preservatives for improving the shelf-life of the kit.

According to another aspect of the present invention, there is provided a kit for preventing or treating a laminin-associated disease or condition.

Such a kit can include, for example, at least one container including at least one of the above described antibodies comprising an antigen recognition domain to QSOX1 and an additional therapeutic agent packed in another container (e.g., chemotherapeutic agents, hormonal therapeutic agents, radiotherapeutic agents, anti-proliferative agents). According to another embodiment, the therapeutic agent (e.g., antibody comprising an antigen recognition domain to QSOX1) and the additional therapeutic agent are packed in the same container. The kit may also include appropriate buffers and preservatives for improving the shelf-life of the kit.

### A Method for Improving Core Packing at the Variable Light-Heavy (vL-vH) Chain Interface

To address the gaps still plaguing contemporary protein design approaches, as discussed in the introductory section hereinabove, the present inventors have developed a protein design strategy that affords sequences of proteins having stable networks of interacting residues at the core packing at the vL-vH interface and selects a small set of diverse designs amenable to low-throughput screening. This design paradigm and practical strategy, and the corresponding computational tools and methods provided herein, addresses the high density of the vL-vH interface by computationally modeling, relaxing, and ranking according to computed energy, a large number of alternative combinations of multipoint mutations and choosing a set of diverse multipoint mutants for experimental testing. Optionally, the protein design strategy may further include the use of PROSS that addresses stability-threshold effects, by first designing a stable antibody scaffold. Note, that the PROSS stability design algorithm very rarely introduces mutations in dense regions of a protein, such as the vL-vH interface. Hence, PROSS and vL-vH interface design can collaborate to yield stable proteins.

As presented herein, starting from exemplary antibody for demonstrative purpose, the method provided herein was used to design dozens of corresponding vL-vH pairs that exhibited improvements of core packing at the vL-vH interface. The robustness and effectiveness of the herein-presented strategy, can be combined with the previously provided method, implemented publicly available protein-stabilization platform “PROSS” (see, U.S. Patent Application Publication No. 2017/0032079 and WO 2017/017673, each of which is incorporated herein by reference as if fully set forth herein; and e.g., www(dot)pross.weizmann.ac.il/).

Main differences between PROSS, and the method provided herein and implemented in AbLift, is that PROSS designs mutations that mostly contribute additively to protein energy and are therefore usually not interdependent. This implies, as is generally observed in designed variants, that PROS S rarely introduces mutations to densely interacting regions of a protein, such as the vL-vH interface or other protein-protein interfaces. This distinction is of paramount importance: Since there are many positions in any protein open to design of stable variants (>90% of the protein is not directly related to function), PROSS looks only for the safest combinations of mutations, using a combinatorial design algorithm that assumes that the backbone stays fixed and results in a combination of mutations with a mostly additive effect on stability. In contrast, AbLift work in the regions of the protein system where positions are highly interdependent (for instance, the binding site between two chains, such as the vL-vH interface). In such structural regions, there are fewer allowed mutations (<=10% of the protein and very high conservation due to functional constraint) and almost all positions are dependent on one-another so there are almost no “safe” combinations of mutations, in which each mutation impacts activity in an additive way; they are all potentially deleterious, and indeed experiments show that these regions are incredibly sensitive to mutation, let alone multipoint mutations. Therefore, in the method provided herein, and implemented as the exemplary procedure AbLift, the tolerated sequence space is identified firstly, using more relaxed settings (energetic stability threshold) than PROSS, so as to enable mutations even in conserved positions, and secondly enumerates all of the possible combinations, which are kept at manageable numbers to enable effective computation. In each instance of a multipoint mutant generated by the method provided herein (AbLift), the backbone is allowed to change conformation, thereby allowing mutations, including small-to-large mutations that are considered very difficult for computational design and even combinations of small-to-large mutations. All of the enumerated multipoint mutants are then ranked by energy to ensure that only stable, pre-organised networks of mutations are selected. It has been surprisingly noticed by the inventors of the present invention, that there are often hundreds or even thousands of sequences with lower energies (more stable) than the wild type or the original/starting sequence, which has not been seen by applying straightforward combinatorial design simulations or in PROSS results. Thus, the method provided herein is based on a rigorous sampling of sequence space with fewer assumptions on the rigidity of the protein or on the additive contribution of mutations to function or stability.

FuncLib is a computational method closely related to AbLift. FuncLib is fine-tuned to generate a family of enzyme variants exhibiting a diverse repertoire of enzymatic activity, compared to an original enzyme. While FuncLib and AbLift share many computational components, the main difference between the two exemplary implementations of the protein design method provided herein, is that FuncLib is mainly applied to enzyme active sites, which are solvent exposed and therefore potentially still tolerant to mutation, whereas AbLift is applied to the interface between two protein chains (e.g., light/heavy chain interface in antibodies). This chain interface region is as tightly packed as a protein core, and therefore potentially less tolerant to mutation. It is noted herein that PROSS, the previously provided method, typically fails to find mutations in such regions, and AbLift is seen to readily find hundreds of multipoint combinations with improved energy (stability and preorganization).

Hence, the method provided herein (AbLift) deals with the problem of how to find favourable multipoint mutants among interdependent positions in highly conserved regions—an outcome that PROS S explicitly tries to avoid, other computational design in general typically fail in, and experimental in vitro evolution strategies often require multiple iterative step-by-step screening in order to achieve.

Thus, according to an aspect of some embodiments of the present invention, there is provided a method for computationally designing a library of antibodies (polypeptides), stemming from a template/original antibody (original polypeptide chain), wherein members of this library exhibit significant improvements in the vL-vH interaction, compared to the template/original antibody.

In terms of parameter values and Rosetta energy units, the more relaxed energetic stability threshold used in AbLift includes PSSM score ≥−2 or −1 and ΔΔG score ≤+1, +2, +3, +4, +5, or +6, compared to the energetic stability threshold used in PROSS, which includes PSSM score ≥0 and ΔΔG score ≤−0.45, −0.9, −2.0, −3.0, or −4.0.

For the demonstration of the method, the antibody with a publically available crystal structure, anti-human QSOX-1 antibody, h492.1, was selected. The method presented herein was effectively used to provide modified polypeptide chains, starting with an original polypeptide chain, such as found in a corresponding wild type protein or a previously engineered/designed variant, wherein several amino acid residues in the original polypeptide chains have been substituted such that a protein expressed to have the modified polypeptide chains (a variant protein) exhibits improved catalytic activity with respect to a certain substrate, as well as structural stability, compared to the wild type protein. The term “variant”, as used herein, refers to a designed protein obtained by employing the method presented herein. Herein and throughout, the terms “amino acid sequence” and/or “polypeptide chain” are used also as a reference to the protein having that amino acid sequence and/or that polypeptide chain; hence the terms “original amino acid sequence” and/or “original polypeptide chain” are equivalent or relate to the terms “original protein” and “wild type protein”, and the terms “modified amino acid sequence” and/or “modified polypeptide chain” and/or “designed polypeptide” are equivalent or relate to the terms “designed protein” and “variant”.

In some embodiments, the original polypeptide chain, or the original protein, is naturally occurring (wild type; WT) or artificial (man-made non-naturally occurring), or a designed polypeptide chain, namely a product of a computational method, such as PROSS.

In the context of some embodiments of the present invention, the term “designed” and any grammatical inflections thereof, refers to a non-naturally occurring sequence or protein.

In the context of some embodiments of the present invention, the term “sequence” is used interchangeably with the term “protein” when referring to a particular protein having the particular sequence.

According to an aspect of some embodiments of the present invention, there is provided a method of computationally designing a modified polypeptide chain starting from an original polypeptide chain.

### Method Requirements and Input Preparation

The basic requirements for implementing the method for designing modified polypeptide chains for activity diversification include:

availability of structural information pertaining to the original polypeptide chain, such as obtained from an experimentally determined crystal structure of the original polypeptide chain, or a crystal structure of a close homolog thereof, having at least 30-60% amino acid sequence identity, or computationally derived structural information based on an experimentally determined structure of a close homolog thereof;

optional availability of experimental mutation analysis, either point mutations, combinations of mutations, or deep mutational scanning; and

availability of sequence data derived from several qualifying homologous proteins, whereas the criteria for a qualifying homologous sequence are described below. In some cases of low availability of homologous proteins, the method utilizes a unique approach for selecting qualifying homologous sequences, as described below.

In the context of embodiments of the present invention, the term “% amino acid sequence identity” or in short “% identity” is used herein, as in the art, to describe the extent to which two amino acid sequences have the same residues at the same positions in an alignment. It is noted that the term “% identity” is also used in the context of nucleotide sequences.

### Structural Data Preparation

According to some embodiments of the invention, the structural information is a set of atomic coordinates of the original polypeptide chain. This set of atomic coordinates is referred to herein as the “template structure”, which is used in the method as discussed below. In some embodiments, the template structure is a crystal structure of the original polypeptide chain, and in some embodiments the template structure is a computationally generated structure based on a crystal structure of a close homolog (more than 30-60% identity) of the original polypeptide chain, wherein the amino acid sequence of the original polypeptide chain has been threaded thereon and subjected to weighted fitting to afford energy minimization thereof, as these are discussed below.

In cases where the protein of interest is an oligomer (having several polypeptide chains), the chain of interest, or the original polypeptide chains to be modified, is defined in the template structure. In the case of hetero-oligomers, it is required to select the chain that will undergo the sequence design procedure or to subject both chains to simultaneous design.

### Structure Refinement

According to some embodiments, prior to its use in the method presented herein, the template structure is optionally subjected to a global energy minimization, afforded by weighted fitting thereof, as discussed below.

According to some embodiments of the present invention, the template structure is optionally refined by energy minimization prior to using its coordinates, while fixing the conformations of key residues, as defined hereinbelow. Structure refinement is a routine procedure in computational chemistry, and typically involves weight fitting based on free energy minimization, subjected to rules, such as harmonic restraints.

The term “weight fitting”, according to some embodiments of any of the embodiment of the present invention, refers to a one or more computational structure refinement procedures or operations, aimed at optimizing geometrical, spatial and/or energy criteria by minimizing polynomial functions based on predetermined weights, restraints and constrains (constants) pertaining to, for example, sequence homology scores, backbone dihedral angles and/or atomic positions (variables) of the refined structure. According to some embodiments, a weight fitting procedure includes one or more of a modulation of bond lengths and angles, backbone dihedral (Ramachandran) angles, amino acid side-chain packing (rotamers) and an iterative substitution of an amino acid, whereas the terms “modulation of bond lengths and angles”, “modulation of backbone dihedral angles”, “amino acid side-chain packing” and “change of amino acid sequence” are also used herein to refer to, inter alia, well known optimization procedures and operations which are widely used in the field of computational chemistry and biology. An exemplary energy minimization procedure, according to some embodiments of the present invention, is the cyclic-coordinate descent (CCD), which can be implemented with the default all-atom energy function in the Rosetta™ software suite for macromolecular modeling. For a review of general optimization approaches, see for example, “Encyclopedia of Optimization” by Christodoulos A. Floudas and Panos M. Pardalos, Springer Pub., 2008.

According to some embodiments of the present invention, a suitable computational platform for executing the method presented herein, is the Rosetta™ software suite platform, publically available from the “Rosetta@home” at the Baker laboratory, University of Washington, U.S.A. Briefly, Rosetta™ is a molecular modeling software package for understanding protein structures, protein design, protein docking, protein-DNA and protein-protein interactions. The Rosetta software contains multiple functional modules, including RosettaAbinitio, RosettaDesign, RosettaDock, RosettaAntibody, RosettaFragments, RosettaNMR, RosettaDNA, RosettaRNA, RosettaLigand, RosettaSymmetry, and more.

Weight fitting, according to some embodiments, is effected under a set of restraints, constrains and weights, referred to as rules. For example, when refining the backbone atomic positions and dihedral angles of any given polypeptide segment having a first conformation, so as to drive towards a different second conformation while attempting to preserve the dihedral angles observed in the second conformation as much as possible, the computational procedure would use harmonic restraints that bias, e.g., the Ca positions, and harmonic restraints that bias the backbone-dihedral angles from departing freely from those observed in the second conformation, hence allowing the minimal conformational change to take place per each structural determinant while driving the overall backbone to change into the second conformation.

In some embodiments, a global energy minimization is advantageous due to differences between the energy function that was used to determine and refine the source of the template structure, and the energy function used by the method presented herein. By allowing changes to occur in backbone conformation and in rotamer conformation through minimization, the global energy minimization relieves small mismatches and small steric clashes, thereby lowering the total free energy of some template structures by a significant amount.

In some embodiments, energy minimization may include iterations of rotamer sampling (repacking) followed by side chain and backbone minimization. An exemplary refinement protocol is provided in Korkegian, A. et al., Science, 2005. In some embodiments, energy minimization may include more substantial energy minimization in the backbone of the protein.

As used herein, the terms “rotamer sampling” and “repacking” refer to a particular weight fitting procedure wherein favorable side chain dihedral angles are sampled, as defined in the Rosetta software package. Repacking typically introduces larger structural changes to the weight fitted structure, compared to standard dihedral angles minimization, as the latter samples small changes in the residue conformation while repacking may swing a side chain around a dihedral angle such that it occupies an altogether different space in the protein structure.

In some embodiments, wherein the template structure is of a homologous protein, the query sequence is first threaded on the protein's template structure using well established computational procedures. For example, when using the Rosetta software package, according to some embodiments of the present invention, the first two iterations are done with a “soft” energy function wherein the atom radii are defined to be smaller. The use of smaller radius values reduces the strong repulsion forces resulting in a smoother energy landscape and allowing energy barriers to be crossed. The next iterations are done with the standard Rosetta energy function. A “coordinate constraint” term may be added to the standard energy function to allow substantial deviations from the original Ca coordinates. The coordinate constraint term behaves harmonically (Hooke's law), having a weight ranging between about 0.05-0.4 r.e.u (Rosetta energy units), depending on the degree of identity between the query sequence and the sequence of the template structure. During refinement, key residues are only subjected to small range minimization but not to rotamer sampling.

### Sequence Data Preparation

Once an original polypeptide chain has been identified, and a corresponding template structure has been provided, the method requires assembling a database of qualifying homologous amino acid sequences related to the amino acid sequence of the original polypeptide chain. The amino acid sequence of the original polypeptide chain can be extracted, for example, from a FASTA file that is typically available for proteins in the protein data bank (PDB), or provided otherwise. The search for qualifying homologous sequences is done, according to some embodiments of the present invention, in the non-redundant (nr) protein database, using the sequence of the original polypeptide chain as a search query. Such nr-database typically contains manually and automatically annotated sequences and is therefore much larger than databases that contain only manually annotated sequences.

A non-limiting examples of protein sequence databases include INSDC EMBL-Bank/DDBJ/GenBank nucleotide sequence databases, Ensembl, FlyBase (for the insect family Drosophilidae), H-Invitational Database (H-Inv), International Protein Index (IPI), Protein Information Resource (PIR-PSD), Protein Data Bank (PDB), Protein Research Foundation (PRF), RefSeq, Saccharomyces Genome Database (SGD), The Arabidopsis Information Resource (TAIR), TROME, UniProtKB/Swiss-Prot, UniProtKB/Swiss-Prot protein isoforms, UniProtKB/TrEMBL, Vertebrate and Genome Annotation Database (VEGA), WormBase, the European Patent Office (EPO), the Japan Patent Office (JPO) and the US Patent Office (USPTO).

A search in an nr-database yields variable results depending on the search query (amino-acid sequence of the original polypeptide chain). For proteins with lacking sequence data, results may include less than 10 hits. For proteins common to all life kingdoms the results may include thousands of hits. For most proteins hundreds to thousands of hits are expected upon search in an nr-database. In all databases, including an nr-database and despite its name, there may be redundancy to some extent, and hits may be found in groups of identical sequences. The redundancy problem is addressed during the sequence data editing.

In some embodiments of the invention, the obtained sequence data is optionally filtered and edited as follows:

(a) Redundant sequences are clustered into a single representative sequence. The clustering is carried out with a predetermined threshold, For example, a threshold of 0.97 means that all sequences that share at least 97% identity among themselves are clustered into a single representative sequence that is the average of all the sequences contributing to the cluster;

(b) Sequences for which the alignment length is less than a predetermined threshold (e.g., 60%) of the search query length are excluded; and

(c) Sequences that exhibit less than about 28% to 34% identity cutoff, for example, with respect to the search query are excluded, following guidelines such as provided elsewhere [Rost, B., Protein Eng, 1999, 12(2):85-94].

The exact choice of the minimal identity parameter depends on the richness of the sequence data. Hence, according to some embodiments of the invention, if the number of sequence hits afforded under a strict threshold is about 50 or less, a less strict threshold may be used (lower % identity). The effect of threshold tuning of the identity parameter is demonstrated in the design of a phosphotriesterase from Pseudomonas diminuta, where lowering the threshold from 30% to 28% identity increased the number of qualifying homologous sequences from 45 to 95.

In some embodiments of the invention, the cutoff for electing qualifying homologous sequences for a multiple sequence alignment is more than 20%, 25%, 30%, 35%, 40%, or more than 50% identity with respect to the original polypeptide chain.

It is noted that the method is not limited to any particular sequence database, search method, identity determination algorithm, and any set of criteria for qualifying homologous sequences. However, the quality of the results obtained by use of the method depends to some extent on the quality of the input sequence data.

Once an assembly of qualifying homologous sequences is obtained, a multiple sequence alignment (MSA) is generated, typically by using a designated multiple sequence alignment algorithm, such as that implemented in MUSCLE [Edgar, R. C., Nucleic Acids Res, 2004, 32(5): 1792-1797]. Alternatively, a Basic Local Alignment Search Tool (BLAST) can be used to generate MSA files.

### Cases of Low Availability of Homologous Proteins

Generally, adding sequences exhibiting a % identity below 20% to a MSA having dozens of homologous sequences of higher % identity may contribute diversity to the alignment; however, adding such kind of low % identity sequences increases the risk of errors (false positives) significantly while not necessarily improving diversity by much, since most of this diversity will probably be covered by the high homology sequences that were already part of the MSA. On the other hand, when the protein of interest is poorly represented in the sequence database, using a low % identity homolog becomes an advantage rather than a risk.

In some cases the protein of interest is poorly represented in the currently available protein sequence databases in terms of the number of non-redundant homologous sequences. For example, in case that a sequence homology search finds only one homologous sequence having 60% sequence identity to the protein of interest, that means that the method is limited to zero amino acid substitutions in 60% of the sequence positions, and out of the remaining 40% it would have been difficult to identify a position with more than few amino acid alternatives.

In such cases, the present inventors have envisioned several scenarios where standard sequence homology search methods might result in low sequence diversity within the space of homologous sequences (e.g., less than 50%, less than 40%, less than 30%, less than 25% (the “twilight zone”) or less than 20% sequence identity with respect to the amino acid sequence of the protein of interest). An example for such a scenario is where the fold of the protein of interest (the target protein, also referred to herein as the original polypeptide chain) is unique or phylogenetically restricted to particular genera or phyla, or the protein function has emerged in recent millennia and the protein of interest therefore has few homologues. It was envisioned by the present inventors that in such or other cases of low sequence diversity, the following steps could be taken to increase the sequence diversity used by presently provided method, while minimizing the risk of introducing unrelated sequences.

An exemplary sub-algorithm for treating such cases is described in U.S. Patent Application Publication No. 2017/0032079, which is incorporated herein by reference. The general rational behind this sub-algorithm is to increase the number of homologous sequences in the MSA as much as possible while minimizing the risk of including non-related sequences; for example, accounting for the fact that the fold of the protein of interest is unique and/or phylogenetically distant from typical organisms interrogated by sequencing efforts.

Step 1: search for low-sequence identity homologous sequences (e.g., less than 50%, less than 40%, less than 30%, less than 25% or less than 20% sequence identity; preferably less than 30% identity) in any given sequence database by using an algorithm that specializes in detection of distant homologues (e.g., CSI-BLAST; see, PMIDs: 19234132, 18004781);

Step 2: cluster the results from Step 1 using a clustering threshold 90-100% (see, e.g., PMID: 11294794);

Step 3: remove sequences with coverage below 40% relative to that of the original polypeptide chain (protein of interest), and sequence identity of less than 15%;

Step 4: inspect the annotation and source organism of each sequence in the list resulting from Step 3, and exclude sequences that have a high chance of being false positives. Non-limiting examples are hits that have no molecular-function annotation (typically these are annotated as “hypothetical protein”), sequences from genera or phyla other than the protein of interest's genus or phylum, or proteins that are annotated with functions that are different from the function of the protein of interest;

Step 5 Exclude sequences that have more than 5%, more than 4%, more than 3%, more than 2%, more than 1%, or more than 0.5% gaps (insertions or deletions, known by the acronym INDELs), preferably less than 5% gaps in a pairwise alignment with the original polypeptide chain (see, e.g., PMID: 18048315);

Step 6: Combine sequences resulting from Step 5 with high sequence identity sequences (i.e., more than 30% sequence identity to the protein of interest) that were collected and processed using any sequence identity search protocol, and generate a multiple-sequence alignment (MSA). This MSA can then be used as input by the method presented herein even if it contains few (less than 3-10) sequences.

Following is a more specific yet non-limiting example:

Step I: Use the CSI-BLAST search algorithm instead of BLASTP to identify homologs. The use of an alternative sequence search algorithm to find distant homologues, such as using CSI-BLAST (context-specific iterative BLAST) with 3 iterations instead of BLASTP is advantageous in some cases since CSI-BLAST constructs a different substitution matrix to calculate alignment scores. The CSI-BLAST matrix is context specific (i.e., each position probabilities depend also on 12 neighboring amino acids), thus it finds 50% more homologous sequences than BLAST at the same error rate. The iterative use means that this process is repeated and at the end of each round the substitution matrix is updated according the sequence information from homologues collected up to that point.

Step II: Use minimal sequence identity thresholds of 19% and 15% for strict and permissive alignments respectively. Lowering the minimal sequence identity threshold to 15% (permissive alignment) and 19% (strict alignment) while using BLASTP may be meaningless since BLASTP is tuned to find sequences with higher sequence identity to the target. Secondly, these thresholds are chosen according to the results obtained from the CSI-BLAST search; hence these thresholds are set after the CSI-BLAST search and depend on outcome; specifically, the thresholds may need to be adjusted to obtain more true positive or fewer false positive hits, where true positive are hits with a functional annotation and phylogenetic origin that correspond to the requirements of Step III, below.

Step III: Exclude sequences from genera or phyla other than the one corresponding to the protein of interest if it is expected that protein target's fold or function are unique to the genus of phylum of the target protein. If this expectation holds, proteins from genera and phyla outside those of the target protein are likely to be false-positive hits; that is, proteins that adopt different folds or function.

Step IV: Use an INDEL fraction of up to 1% for sequences sharing below 19% sequence identity, in pairwise alignment with the query. In the treatment of gaps/INDELs the CSI-BLAST pairwise alignment INDELS fraction may be required to be up to 1% for sequence with minimal % identity below 19%. The rationale is that for low-homology sequences sharing such a small sequence identity to the query, the risk of inserting false positives in the MSA is too high, but a small INDEL fraction indicates that these are likely to be true hits.

Step V: Use sequence coverage threshold for hits relative to the target protein in the alignment to 50%. It is likely that all the sequences that passed the criteria set forth in Steps II, III and IV will exhibit a coverage of more than 50%; however, if the coverage threshold is set to 60%, as typically practiced in the art, most of the sequences would be filtered out.

Step VI: Generate MSA for the remaining sequences as typically practiced in the art.

### Variable Loop Regions

BLAST algorithms may provide results that include sequences with different lengths. The differences typically stem from different lengths in loop regions, and loops with different lengths may reflect different biochemical context. As a result, MSA columns representing loop positions may contain aligned residues from loops with different length, thus possibly degrading the data with information from different biochemical context, possibly irrelevant to the biochemical context of the protein of interest. A BLAST hit may therefore contain relevant information at some positions while containing non-relevant information in other positions. To minimize the level of irrelevant sequence information for each loop, the secondary structure of the original protein is identified and a context specific sub-MSA file is created for each loop region, and the sub-MSA contains only loop sequences with the same length.

Secondary structure identification is done through identification of hydrogen bond patterns in the structure and this is termed “dictionary of protein secondary structure” (DSSP). There are several software packages available that offer such analysis, such as, for example, a Rosetta™ module for loop identification.

The output of the secondary structure identification procedure is typically a string (i.e., an output string) that has the same length as the template structure, wherein each character represents a residue in a secondary structure element that may be either H, E or L, denoting an amino acid forming a part of either an α-helix, a β-sheet or a loop.

According to some embodiments of the invention, the amino acid sequence of the loop regions in the structure of the original protein is processed as follows:

(a) Loops in the template structure are identified by automatic or manual inspection of a structure model, and/or by any secondary-structure analyzing algorithms.

(b) The positions representing each loop on the output string are determined including loop stems (two additional amino acids at each end of the loop). To account for the stems, two positions are added to each of the loop's ends, unless the loop is at one of the main-chain termini. According to some embodiments of the invention, it is advantageous to include the stems in the loop definition since stems anchoring different loops may potentially exhibit different conformations and form different contacts among themselves or with the loop residues, and it is advantageous that the sequence data used as input in the method presented would represent that.

For example, if the secondary structure output string is:
LLLHHHHHHHLLLLLHHHHHLLLEEEE
   then the loop regions are defined at positions 1-5, 9-17 and 19-25 (bold characters).

(c) The positions that represent each loop are identified in the query sequence in the MSA. The loop positions in the MSA may be different than the loop positions in the original string from the previous step since in the MSA the query is aligned to other sequences and may therefore contain both amino acid characters and hyphens, representing gaps.

(d) After the loop positions were located on the query sequence in the MSA, a character pattern is defined for each loop. For example, a pattern may comprise “X” character to represent an amino acid and “-” (hyphen) to represent a gap.

(e) Lastly, a context specific sub-MSA file is generated for each loop excluding all sequences that do not share the same character pattern for that loop, namely context specific sub-MSA contains sequences wherein the loop has the same length, gaps included.

For example, positions 4-10 in a hypothetical original protein are recognized as a loop with the hypothetical sequence “APTESVV” (SEQ ID NO: 120) including stems. The loop is identified on the query protein in the MSA file and the pattern is found to be “A--PTESVV”. The context specific sub-MSA file that will be generated for this loop with all the sequences in the MSA file will contain the pattern “X--XXXXX”.

Thus, according to some embodiments of the present invention, for loop regions, the sequence alignment comprises amino acid sequences having sequence length equal to a corresponding loop in the original polypeptide chain. Accordingly, sequence alignments, which are relevant in the context of loop regions, are referred to herein as “context specific sub-MSA”.

### Rules for Substitutions

The method calls for identification of substitutable residues. The selection of substitutable residues may rely on expert-guided decision on positions to mutate. These positions are typically positions in the light-to-heave chain interface.

In some embodiments of the present invention, a set of restraints, constrains and weights are used as rules that govern some of the computational procedures. In the context of some embodiments of the present invention, these rules are applied in the method presented herein to determine which of the positions in the original polypeptide chain will be allowed to permute (be substituted), and to which amino acid alternative. These rules may also be used to preserve, at least to some extent, some positions in the sequence of the original polypeptide chain.

One of the rules employed in amino acid sequence alterations stem from highly conserved sequence patterns at specific positions, which are typically exhibited in families of structurally similar proteins. According to some embodiments of the present invention, the rules by which a substitution of amino acids is dictated during a sequence design procedure include position-specific scoring matrix values, or PSSMs.

A “position-specific scoring matrix” (PSSM), also known in the art as position weight matrix (PWM), or a position-specific weight matrix (PSWM), is a commonly used representation of recurring patterns in biological sequences, based on the frequency of appearance of a character (monomer; amino acid; nucleic acid etc.) in a given position along the sequence. Thus, PSSM represents the log-likelihood of observing mutations to any of the 20 amino acids at each position. PSSMs are often derived from a set of aligned sequences that are thought to be structurally and functionally related and have become widely used in many software tools for computational motif discovery. In the context of amino acid sequences, a PSSM is a type of scoring matrix used in protein BLAST searches in which amino acid substitution scores are given separately for each position in a protein multiple sequence alignment. Thus, a Tyr-Trp substitution at position A of an alignment may receive a very different score than the same substitution at position B, subject to different levels of amino acid conservation at the two positions. This is in contrast to position-independent matrices such as the PAM and BLOSUM matrices, in which the Tyr-Trp substitution receives the same score no matter at what position it occurs. PSSM scores are generally shown as positive or negative integers. Positive scores indicate that the given amino acid substitution occurs more frequently in the alignment than expected by chance, while negative scores indicate that the substitution occurs less frequently than expected. Large positive scores often indicate critical functional residues, which may be active site residues or residues required for other intermolecular or intramolecular interactions. PSSMs can be created using Position-Specific Iterative Basic Local Alignment Search Tool (PSI-BLAST) [Schïffer, A. A. et al., Nucl. Acids Res., 2001, 29(14), pp. 2994-3005], which finds similar protein sequences to a query sequence, and then constructs a PSSM from the resulting alignment. Alternatively, PSSMs can be retrieved from the National Center for Biotechnology Information Conserved Domains Database (NCBI CDD) database, since each conserved domain is represented by a PSSM that encodes the observed substitutions in the seed alignments. These CD records can be found either by text searching in Entrez Conserved Domains or by using Reverse Position-Specific BLAST (RPS-BLAST), also known as CD-Search, to locate these domains on an input protein sequence.

In the context of some embodiments of the present invention, a PSSM data file can be in the form of a table of integers, each indicating how evolutionary conserved is any one of the 20 amino acids at any possible position in the sequence of the designed protein. As indicated hereinabove, a positive integer indicates that an amino acid is more probable in the given position than it would have been in a random position in a random protein, and a negative integer indicates that an amino acid is less probable at the given position than it would have been in a random protein. In general, the PSSM scores are determined according to a combination of the information in the input MSA and general information about amino acid substitutions in nature, as introduced, for example, by the BLOSUM62 matrix [Eddy, S. R., Nat Biotechnol, 2004, 22(8), pp. 1035-6].

In general, the method presented herein can use the PSSM output of a PSI-BLAST software package to derive a PSSM for both the original MSA and all sub-MSA files. A final PSSM input file, according to some embodiments of the present invention, includes the relevant lines from each PSSM file. For sequence positions that represent a secondary structure, relevant lines are copied from the PSSM derived from the original full MSA. For each loop, relevant lines are copied from the PSSM derived from the sub-MSA file representing that loop. Thus, according to some embodiments of the present invention, a final PSSM input file is a quantitative representation of the sequence data, which is incorporated in the structural calculations, as discussed hereinbelow.

According to some embodiments of the present invention, MSA and PSSM-based rules determine the unsubstitutable positions and the substitutable positions in the amino acid sequence of the original polypeptide chain, and further determine which of the amino acid alternatives will serve as candidate alternatives in the single position scanning step of the method, as discussed hereinbelow.

### Key Residues

The method, according to some embodiments of the present invention, allows the incorporation of information about the original polypeptide chain and/or the wild type protein. This information, which can be provided by various sources, in incorporated into the method as part of the rules by which amino acid substitutions are governed during the design procedure. Albeit optional, the addition of such information is advantageous as it reduces the probability of the method providing results which include folding- and/or function-abrogating substitutions. In the examples presented in the Example section below, valuable information about activity has been employed successfully as part of the rules.

The term “key residues” refer to positions in the designed sequence that are defined in the rules as fixed (invariable), at least to some extent. Sequence positions, which are occupied by key residues optionally, constitute a part of the unsubstitutable positions.

Information pertaining to key residues can be extracted, for example, from the structure of the original polypeptide chain (or the template structure), or from other highly similar structures when available. Exemplary criteria that can assist in identifying key residues, and support reasoning for fixing an amino-acid type or identity at any given position, include:

In the previous provided protein stabilization design method, PROSS, when used to provide stabilized protein variants, the key residues are selected within a radius of about 5-8 A around a functional site, as may be inferred from complex crystal structures comprising a substrate, a substrate analog, an inhibitor and the like. Similarly, when using PROSS to provide stabilized metal binding proteins, key residues are selected within about 5-8 A around a metal atom. Other key residues may be designated in protein interface that involves the chain of interest in oligomers, as interacting chains are oftentimes involved in dimerization interfaces, binding ligands or protein-substrates interactions. Likewise, key residues may be designated within a certain distance from DNA/RNA chains interacting with the protein of interest, within a certain distance from an epitope region, and the likes.

It is noted that the shape and size of the space within which key residues are selected is not limited to a sphere of a radius of 5-8 A; the space can be of any size and shape that corresponds to the sequence, function and structure of the original protein. It is further noted that specific key residues may be provided by any external source of information (e.g., a researcher).

In the context of the present invention, key residues are selected sparingly (≤10 positions, and more typically 0-3 positions), even and particularly in and around regions of vL-vH interface the method is attempting to diversify or improve. This strategy allows the activity-determining regions to diversify while the stability of the protein is not sacrificed.

When the template structure, the PSSM file (which is based on the full MSA and any optional context specific sub-MSA), and the identification of key residues, unsubstitutable positions and the substitutable positions are provided, the method presented herein can use these data to provide the modified polypeptide chain starting from the original polypeptide chain.

### Main Method Steps

The objective of the method provided herein (AbLift) is to design a small set of stable multipoint vL-vH interface mutants suitable for low-throughput experimental testing. The design strategy is general and can be applied, in principle, to any natural or designed protein, using its molecular structure and a diverse set of homologous sequences and particularly, to other protein-protein interfaces or to other densely packed regions of a protein, including the core of any protein.

According to some embodiments of the present invention, the method presented herein includes a step that determines which of the positions in the amino-acid sequence of the original polypeptide chain will be subjected to amino-acid substitution and which amino acid alternatives will be assessed. (referred to herein as substitutable positions), and in which positions in the amino acid sequence of the original polypeptide chain the amino-acid will not be subjected to amino-acid substitution (referred to herein as unsubstitutable or fixed positions).

In a following step, (single position scanning step), a position-specific stability score is given to each of the allowed amino acid alternatives at each substitutable position. In antibody design, the residues at the vL-vH interface were defined to be designed by visual examination of the molecular structures. Evolutionary conservation scores were computed from PSSMs and ΔΔG values were computed essentially as described previously [Goldenzweig, A. et al. Mol Cell., 2016, 63(2), pp. 337-346]. Tolerated amino acid identities at the active site of PTE were filtered according to the following thresholds: PSSM≥−2 and ΔΔG≤+6 R.e.u.

It is noted that the detailed description of the method presented herein is using some terms, units and procedures with are common or unique to the Rosetta™ software package, however, it is to be understood that the method is capable of being implemented using other software modules and packages, and other terms, units and procedures are therefore contemplated within the scope of the present invention.

It is also noted that the detailed description of the method presented herein is using the proteins and variables presented in the Examples section, which are not to be seen as limiting in any way, as the method is applicable for any protein and polypeptide chain sequence for which the required data is available.

According to some embodiments of the present invention, the following step of the method is an exhaustive enumeration of all possible combinations of at least 2 or 3 and as many as 5, 6, 7, 8, 9, 10 or more than ten mutations in the original polypeptide chain (e.g. antibody). Each mutant was modeled in Rosetta, including combinatorial sidechain packing, and the backbone and sidechains of all residues were minimized energetically, subject to harmonic restraints on the Ca coordinates of the entire protein (being composed of one polypeptide chain or more). All designed polypeptide chains (designed proteins, or “designs” for short) were ranked according to all-atom energy, and the top-ranked designs were chosen for experimental analysis after removing designs with fewer than two mutations relative to one another.

As stated hereinabove, one of the main differences between PROS S and the method provided herein is the combinatorial design step in PROS S that is being replaced by a comprehensive enumeration step in the instant method. In the exemplary study presented here, small-scale testing of the method provided herein (AbLift) proved sufficient to identify variants that exhibited orders-of-magnitude improvement in mammalian cell expression levels and a large change in thermal stability without a significant change in biological activity. It is anticipated that in other cases, the procedure may result in improvements also to binding affinity and biological activity. The method can therefore be used to rapidly optimize antibodies that are not amenable to high-throughput screening.

A typical antibody variable domain comprises more than 200 amino acids. According to some embodiments of the present invention, the method is implemented effectively for original polypeptide chains that comprise more than 100 amino acids (aa). In some embodiments, the original polypeptide chains comprise more than 110 aa, more than 120 aa, more than 130 aa, more than 140 aa, more than 150 aa, more than 160 aa, more than 170 aa, more than 180 aa, more than 190 aa, more than 200 aa, more than 210 aa, more than 220 aa, more than 230 aa, more than 240 aa, more than 250 aa, more than 260 aa, more than 270 aa, more than 280 aa, more than 290 aa, more than 300 aa, more than 350 aa, more than 400 aa, more than 450 aa, more than 500 aa, more than 550 aa, or more than 600 amino acids.

According to some embodiments of the present invention, the method presented herein provides modified polypeptide chains having more than 2 amino acid substitutions (mutations), more than 3 substitutions, more than 4 substitutions, more than 5 amino acid substitutions, more than 6 substitutions, more than 7 substitutions, more than 8 substitutions, more than 9 substitutions, more than 10 substitutions, more than 11 substitutions, or more than 12 substitutions compared to the starting original polypeptide chain.

### Sequence Space

According to some embodiments of the present invention, after filtering key residues and imposing a free energy acceptance threshold, the number of substitutable positions in a given sequence is greatly reduced, thereby providing a wide yet manageable combinatorial sequence space from which designed sequences can be selected. Thus, the term “sequence space” refers to a set of substitutable positions, each having at least one optional substitution over the original/WT amino acid at the given position.

A sequence space is therefore a result of a certain acceptance threshold; each acceptance threshold produces a different sequence space, where sequence spaces defined by stricter acceptance thresholds are contained within larger sequence spaces defined by more permissive acceptance thresholds. As discussed hereinabove, in order to avoid false positives the acceptance threshold can be small and should be negative, wherein −2 r.e.u is considered to be highly restrictive (strict) and +6 r.e.u is highly permissive. The sequence space obtained by using acceptance threshold of +6 r.e.u will inevitably be larger (permissive) than a sequence space obtained by using acceptance threshold of −2.00 r.e.u (strict). Experimental use of the method presented herein to produce actual proteins has shown that an intermediate acceptance threshold produces an optimal sequence space. In fact, the sequence space is a sub-space of the broader space defined by the PSSM rules.

An exemplary and general means to present a sequence space is in a list of sequence positions based on the wild-type sequence numbering, P₁, P₂, P₃, . . . , Pn, wherein each position is either designated as a key residue, namely an amino acid as found in the WT, AA_{WT}; or a position that can take any one amino acid from a limited list comprising at least one alternative amino acid based on the PSSM and energy minimization analysis, AAₘ, wherein m is a number denoting one of the naturally occurring amino acids, e.g., A=1, R=2, N=3, D=4, C=5, Q=6, E=7, G=8, H=9, L=10, I=11, K=12, M=13, F=14, P=15, S=16, T=17, W=18, Y=19 and V=20 (aa numbering is arbitrary and used herein to demonstrate a general representation of a sequence space.

For example, the sequence space can be presented as:

P₁: AA_{WT}, AA₅, AA₈, and AA₁₂;

P₂: AA_{WT};

P₃: AA_{WT} and AA₁₆;

P₄: AA_{WT}, AA₁, AA₃, AA₆, AA₁₀, and AA₁₄;

P₅: AA_{WT}, AA₄, AA₈, and AA₁₁;

. . .

Pn: AA_{WT}, AAₘ, AAₘ, AAₘ, AAₘ, and AAₘ;

whereas in this general example, P₁ has four alternative amino acids, P₂ is a key residue and so forth.

According to some embodiments of the present invention, the sequence space can be further limited by imposing a stricter acceptance threshold, or expanded by imposing a more permissive acceptance threshold. In general, the value of +2 r.e.u has been found to be adequately permissive; however sequence space based on an acceptance threshold larger than +2 r.e.u (e.g., +6 r.e.u) or based on an acceptance threshold smaller than −2.00 r.e.u (e.g., −2.1 r.e.u) are also contemplated.

In the Examples section that follows below, a sequence space based on acceptance threshold of +6 r.e.u is presented for some of the exemplary proteins on which the method has been demonstrated. Any designed sequence having any choice of any 2 or more substitutions relative to the wild-type/starting sequence that are selected from the presented sequence space, and that exhibits, at least one improved catalytic activity, is contemplated within the scope of the present invention.

It is noted herein that embodiments of the present invention encompass any and all the possible combinations of amino acid alternatives in any given sequence space afforded by the method presented herein (all possible variants stemming from the sequence space as defined herein).

It is further noted that in some embodiments of the present invention, the sequence space resulting from implementation of the method presented herein on an original protein, can be applied on another protein that is different than the original protein, as long as the other protein exhibits at least 30%, at least 40%, or at least 50% sequence identity and higher. For example, a set of amino acid alternatives, taken from a sequence space afforded by implementing the method presented herein on a human protein, can be used to modify a non-human protein by producing a variant of the non-human protein having amino acid substitutions at the sequence-equivalent positions. The resulting variant of the non-human protein, referred to herein as a “hybrid variant”, would then have “human amino acid substitutions” (selected from a sequence space afforded for a human protein) at positions that align with the corresponding position in the human protein. In some embodiments of the present invention, any such hybrid variant, having at least 2 substitutions that match amino acid alternatives in any given sequence space afforded by the method presented herein (all possible variants stemming from the sequence space as defined herein), is contemplated and encompassed in the scope of the present invention.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which, together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Materials and Methods

TABLE 1
The mutated positions and identities in anti-QSOX1 492.1 designs.

| | light chain | | | | heavy chain | | | | | | | | Total score |
|---|---|---|---|---|---|---|---|---|---|---|---|---|---|
| | S43 | Y55 | Q89 | L96 | V37 | G44 | L45 | D58 | F94 | S104 | F105 | A106 | [R.e.u.] |
| h492.1 | S | Y | Q | L | V | G | L | D | F | S | F | A | −905 |
| design 1 | — | H | — | F | — | A | — | — | Y | A | L | — | −916 |
| design 2 | — | P | — | F | — | — | — | E | — | A | — | E | −916 |
| design 3 | A | P | — | — | — | — | — | E | Y | A | L | E | −916 |
| design 4 | P | H | — | — | — | — | — | E | Y | — | — | P | −916 |
| design 5 | P | P | — | F | — | — | — | — | — | — | — | N | −916 |
| design 6 | P | P | — | F | — | — | — | — | — | A | L | E | −917 |
| design 7 | P | P | — | F | — | — | — | E | Y | — | L | E | −916 |
| design 8 | P | P | — | F | — | A | — | — | — | — | Y | Q | −916 |
| design 9 | P | P | — | F | — | A | — | — | Y | A | L | Q | −916 |
| design 10 | P | P | — | F | — | A | — | E | — | — | L | — | −916 |
| design 11 | P | P | H | — | — | — | — | — | — | A | — | Q | −917 |
| design 12 | P | P | H | — | — | A | — | — | Y | — | — | Q | −916 |
| design 13 | P | P | H | F | — | — | — | — | — | — | L | — | −916 |
| design 14 | P | P | H | F | — | — | — | — | Y | A | Y | E | −916 |
| design 15 | P | P | H | F | — | — | — | E | — | — | Y | Q | −916 |
| design 16 | P | v | — | F | — | — | F | — | — | — | Y | Q | −916 |
| design 17 | P | v | — | F | — | A | — | — | — | A | L | — | −917 |
| design 18 | P | v | — | F | F | — | — | — | — | — | — | — | −916 |
| design 19 | P | V | H | — | — | A | — | E | Y | A | L | — | −916 |
| design 20 | P | V | H | F | — | A | — | — | — | — | Y | — | −916 |
| design 21 | P | — | — | — | — | — | — | — | — | — | — | — | N/A |
| design 22 | — | P | — | — | — | — | — | — | — | — | — | — | N/A |
| design 23 | P | P | — | — | — | — | — | — | — | — | — | — | N/A |

Anti-QSOX1 Antibody Production

The coding sequences for variable domains of antibody 492.1 were fused to human antibody constant regions (Tiller et al., 2008 J. Immunol. Methods 329; 112-124). Mutations were introduced by site-directed mutagenesis into the resulting hybrid antibody expression plasmids according to published procedures [Unger, T., Jacobovitch, Y., Dantes, A., Bernheim, R. & Peleg, Y. Applications of the Restriction Free (RF) cloning procedure for molecular manipulations and protein expression. J. Struct. Biol. 172, 34-44 (2010)]. Plasmids were transfected into suspension-adapted suspension-HEK 293F cells. The day before transfection, cells were split to 0.7×10⁶ cells/ml. For parallel expression of the parent hybrid antibody and the 20 variants, transfections were performed using 0.5 μg of each plasmid (heavy and light Ab chains) mixed with 3 μg PEI Max reagent (Polysciences Inc.) and incubated 20 min in 24-well tissue culture trays prior to addition of 1 ml cells per well. Plates were then agitated vigorously in a tissue culture incubator/shaker to prevent cell settling. After 4 days, cultures were transferred to microfuge tubes, and cells were pelleted by centrifugation at 500×g for 10 min. Supernatants were transferred to fresh microfuge tubes, from which aliquots were taken for quantification of antibody expression and activity. For purification of selected Ab designs, transfections were done in 40 ml volumes, and plasmid and PEI Max amounts were scaled up accordingly. Cultures were grown for 6 days, and Ab was purified from supernatant by protein G affinity chromatography (GE Healthcare).

QSOX1 Dot Blot and Western Blot Assays

Relative antibody concentrations were determined from culture supernatants by dot and Western blotting. Blotting was conducted in triplicate for each of two biological replicates. For dot blots, 2 μl of each supernatant was spotted onto nitrocellulose membranes. Membranes were then covered with a blocking solution of PBS containing 0.1% tween (PBS-T) and 5% bovine serum albumin (BSA) and gently agitated for 1 h at room temperature. For western blots, 10 μl of each supernatant was applied with non-reducing gel loading buffer to 10% SDS polyacrylamide gels. After electrophoresis, proteins were transferred to nitrocellulose, and the membranes were incubated in PBS-T with 5% BSA under gentle agitation. For both dot and Western blots, horseradish peroxidase-conjugated antibody recognizing human Fc was added to the blocking solution after the first hour, and incubation/shaking was continued for another 45 min. The membrane was then washed three times for 5 min each with PBS-T, and the blot was developed using SuperSignal™ West Pico (ThermoFisher) chemiluminescent substrate. Dot and band intensities were recorded on a ChemiDoc XRS+ system (Bio-Rad).

QSOX1 Inhibition Assays

QSOX1 inhibition assays were conducted by using DTNB to quantify the remaining DTT after incubation with purified recombinant QSOX1 and HEK293 culture supernatants or purified antibody. Culture supernatants (25 μl) were mixed in a clear, flat-bottom, 96-well plate with 12.5 μl QSOX1 of 40 nM QSOX1, and reactions were initiated by injection of 12.5 μl 600 μM DTT (final concentrations 10 nM QSOX1 and 150 μM DTT). Reactions were stopped after 30 min by adding 150 μl 500 μM DTNB, and absorbance at 412 nm was measured after 5 min in a Tecan microplate reader.

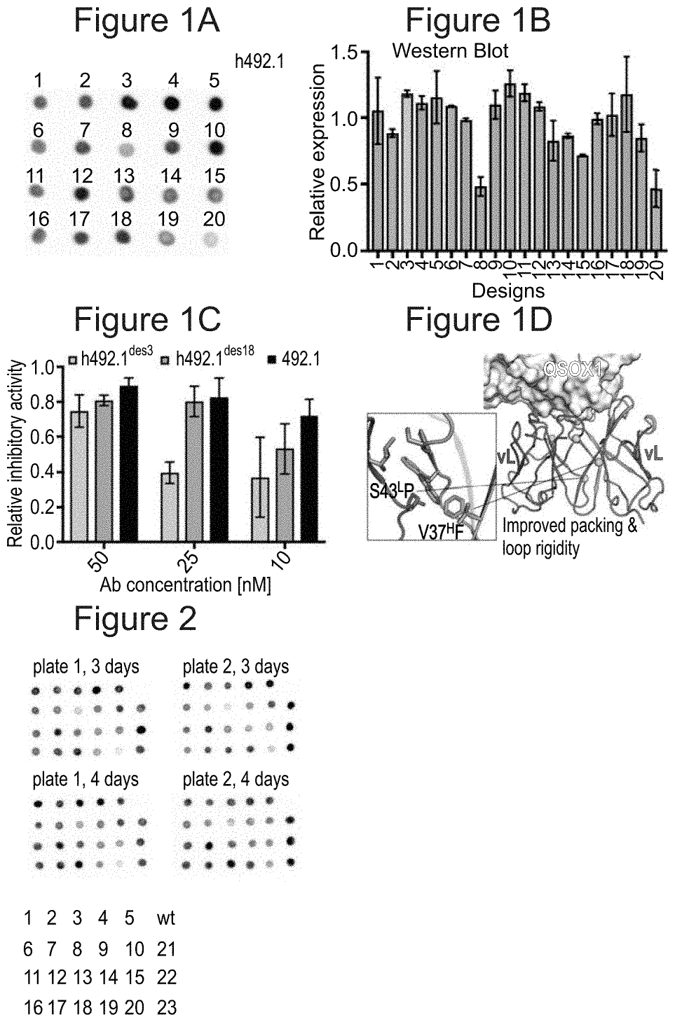

Purified antibody variants were quantified by absorbance at 280 nm after dilution into 6 M guanidine dissolved in PBS, using an extinction coefficient of 187,000 M⁻¹ cm⁻¹. Purified antibodies (12.5 μl) at concentrations of 40 nM, 100 nM, and 200 nM were mixed in a 96-well plate with 12.5 μl 100 nM QSOX1, and reactions were initiated by injection of 25 μl 600 μM DTT (final concentrations 25 nM QSOX1, 300 μM DTT, and 10, 25, or 50 nM antibody). Reactions were stopped after 20 min by adding 150 μl 500 μM DTNB, and absorbance at 412 nm was measured after 5 min. Background-subtracted absorbance readings were normalized relative to the uninhibited and fully inhibited reactions (the latter mimicked by leaving QSOX1 out of the reaction), and results were plotted in FIG. 1C as relative inhibitory activity.

### Example 2

### Design Production and Qualification of Antibodies

The present inventors developed a fully automated design protocol for improving core packing at the vL-vH interface. The design strategy, which is also termed “AbLIFT”, starts by computing a mutational-tolerance map at the vL-vH interface using the approach described above; then exhaustively enumerates multipoint combinations of tolerated mutations; ranks them by energy; and selects low-energy variants for experimental testing. To validate this strategy, the 492.1 antibody (h492.1), which targets human Quiescin Sulfhydryl Oxidase 1 (QSOX1) was selected. QSOX1 is a unique, multi-domain disulfide-catalyst that is overproduced in tumors and is a potential drug target (see Background section). The h492.1 antibody was obtained by fusing the variable domains from the high-affinity (K_{D} approximately 1 nM) QSOX1-inhibiting murine antibody 492.1 onto a human IgG scaffold. Following this fusion, h492.1 could not be expressed to detectable levels in a recombinant cultured human cell system, restricting its further development. An automated computational approach was applied to improve this parameter by improving core packing at the vL-vH interface.

Since h492.1 failed to show detectable expression in HEK293 cell cultures, computational design was initiated from the structure of the murine 492.1 parental antibody in complex with QSOX1 (PDB entry: 4IJ3)⁴⁰ and selected the 20 lowest-energy AbLIFT designs for HEK293 expression screening as IgG1 constructs. Dot blot analysis showed detectable expression levels for all 20 designs, in clear contrast with the lack of detectable expression for h492.1 (FIG. 1A). Expression levels were further quantitated using Western blot, revealing substantial variation in the expression levels among the designs (FIG. 1B). In parallel, the levels of QSOX1 inhibition of the 20 designs were examined, finding that half of the designs showed high levels of QSOX1 inhibition (FIG. 1C). Based on activities and expression levels, designs 3 and 18 were selected for further analysis. These designs were purified and added to QSOX1 activity assays to test for inhibition. The h492.1^{des18} antibody showed comparable inhibitory levels to the parent antibody when provided at equimolar amounts to a typical physiological concentration of QSOX1 (25 nM) as found in human serum (FIG. 1C) [Israel, B. A., Jiang, L., Gannon, S. A. & Thorpe, C. Disulfide bond generation in mammalian blood serum: detection and purification of quiescin-sulfhydryl oxidase. Free Radic. Biol. Med. 69, 129-135 (2014)] that likely resulted from improved packing at the vL-vH interface (FIG. 1D). This analysis demonstrated that h492.1^{des18} almost completely recovered the activity of the parental antibody, while gaining high expression levels (approximately 75 mg/L supernatant).

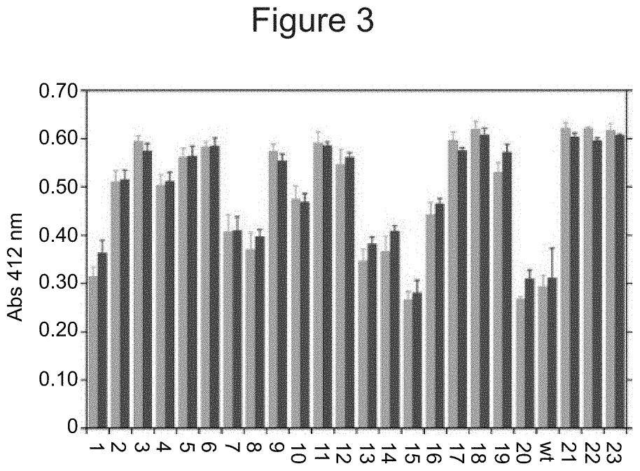

Additional results were obtained with Designs 21, 22 and 23, which contained single proline substitutions or a double substitution (Design 23). The results are shown in FIGS. 2 and 3.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. An isolated antibody comprising an amino acid sequence of a light chain (vL) as set forth in SEQ ID NO: 1 and a heavy chain (vH) as set forth in SEQ ID NO: 2, wherein at least one of said vL and said vH comprises at least one amino acid substitution selected from the group consisting of:

| | |
|---|---|
| 34L | AS |
| 43L | SAPT |
| 46L | LV |
| 55L | YHPV |
| 89L | QH |
| 96L | LF |
| 37H | VF |
| 44H | GAKR |
| 45H | LF |
| 58H | DE |
| 94H | FY |
| 104H | SA |
| 105H | FLY |
| 106H | AENPQ |

wherein said antibody binds QSOX1.

2. An isolated antibody comprising an antigen recognition domain comprising complementarity-determining regions as set forth in GFSLTGYG (CDRHI) (SEQ ID NO: 83), IWGDGRT (CDRH2) (SEQ ID NO: 84) and ASDYYGSGX₁X₂X₃Y, (CDRH3) (SEQ ID NO: 97), QDVSTA (CDRLI) (SEQ ID NO: 86), SAS (CDRL2) (SEQ ID NO: 87) and QQHYSIPX₄T (CDRL3) (SEQ ID NO: 98), wherein X₁, X₂, X₃ and X₄ is any amino acid, wherein said antibody binds QSOX1, wherein when said antibody comprises CDRH3 set forth in SEQ ID NO: 85 said CDRL3 is not as set forth in SEQ ID NO: 88.

3. The antibody of claim 2 wherein said X₁ is selected from the group consisting of S and A.

4. The antibody of claim 2, wherein said X₂ is selected from the group consisting of F, L and Y.

5. The antibody of claim 2, wherein said X₃ is selected from the group consisting of A, E, N, P and Q.

6. The antibody of claim 2, wherein said X₄ is F.

7. The antibody of claim 2, wherein said CDRH3 is selected from the group consisting of ASDYYGSGALEY (SEQ ID NO: 99), ASDYYGSGALAY (SEQ ID NO: 100), ASDYYGSGAFEY (SEQ ID NO: 101), ASDYYGSGALEY (SEQ ID NO: 102), ASDYYGSGSFPY (SEQ ID NO: 103), ASDYYGSGSFNY (SEQ ID NO: 104), ASDYYGSGALEY (SEQ ID NO: 105), ASDYYGSGSLEY (SEQ ID NO: 106), ASDYYGSGSYQY (SEQ ID NO: 107), ASDYYGSGALQY (SEQ ID NO: 108), ASDYYGSGSLAY (SEQ ID NO: 109), ASDYYGSGAFQY (SEQ ID NO: 110), ASDYYGSGSFQY (SEQ ID NO: 111), ASDYYGSGSLAY (SEQ ID NO: 112), ASDYYGSGAYEY (SEQ ID NO: 113), ASDYYGSGSYQY (SEQ ID NO: 114), ASDYYGSGSYQY (SEQ ID NO: 115), ASDYYGSGALAY (SEQ ID NO: 116), ASDYYGSGALAY (SEQ ID NO: 117) and ASDYYGSGSYAY (SEQ ID NO: 118).

8-11. (canceled)

12. The antibody of claim 2, wherein said at least one amino acid substitution is at said position 43L and alternatively or additionally 55L.

13-14. (canceled)

15. The antibody of claim 2, exhibiting an increased expression yield when expressed in HEK293 cells as determined by Western blot and at least about the same QSOX1 inhibitory activity as determined by sulfhydryl oxidase assay as that of an antibody designated m492.1.

16. The antibody of claim 2 set forth in SEQ ID NOs: 8, 48 or 38, 78.

17. The antibody of claim 2, wherein said antibody inhibits QSOX1 activity in mediating laminin incorporation in the basement membrane.

18. The antibody of claim 17, wherein said activity is assayed by at least one of an immunofluorescence (IF) staining assay of an extracellular matrix or a Western blot assay detecting for soluble laminin.

19-20. (canceled)

21. A pharmaceutical composition comprising as an active ingredient the antibody of claim 2 and a pharmaceutically acceptable carrier.

22. A method for preventing or treating a laminin-associated disease or condition in a subject in need thereof, the method comprising administering to the subject a therapeutically effective amount of the pharmaceutical composition of claim 21.

23-27. (canceled)

28. The method of claim 22, wherein said laminin-associated disease or condition is a tumor or fibrosis.

29. The method of claim 28, wherein said tumor is a metastasizing solid tumor.

30. The method of claim 28, wherein said tumor is an adenocarcinoma.

31. The method of claim 28, wherein said tumor is a cancer selected from the group consisting of a prostate cancer, a lung cancer, a breast cancer, a cervical cancer, an urachus cancer, a vaginal cancer, a colon cancer, an esophagus cancer, a pancreatic cancer, a throat cancer, a stomach cancer and a myeloid leukemia.

32-34. (canceled)

35. A method of producing an antibody to QSOX1, the method comprising:
(a) culturing cells expressing an isolated polynucleotide comprising a nucleic acid sequence encoding the antibody of claim 2 under conditions which allow for expression of said vH and/or vL chains;
(b) recovering the vH and/or vL chains from the cells.

36-37. (canceled)


SEQUENCE LISTING (120 sequences)

SEQ ID NO: 1 - protein, 233 aa, Artificial sequence
  Mab 492.1 (Chimeric antibodies human constant regions) light chain amino acid sequence
MGWSCIILFLVATATGVHSDVVMTQTHKFMSTSVGDRVSITCKASQDVSTAVAWYQQKSGQSPKLLIHSASYRYTGVPDRFTGSGSGTDFTFTISSVQAEDLAVYYCQQHYSIPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 466 aa, Artificial sequence
  Mab 492.1 (Chimeric antibodies human constant regions) heavy chain amino acid sequence
MGWSCIILFLVATATGVHCQVQLKQSGPGLVAPSQSLSITCTVSGFSLTGYGVNWVRQSPGKGLEWLGMIWGDGRTDYKSALKSRLSITKDNSKSQVFLKMNSLQTDDTARYFCASDYYGSGSFAYWGQGTLVTVSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 3 - DNA, 321 bp, Artificial sequence
  Design 1 light chain variable domain sequence
gatgtggttatgacccaaacgcataaattcatgagcacctctgtgggcgatcgtgttagtattacgtgtaaggcatcacaggacgtctcgaccgcagtggcctggtatcagcaaaaaagtggtcaatccccgaagctgctgatccacagtgcctcctatcgtcacaccggcgttccggatcgctttaccggctcaggttcgggcacggactttaccttcacgattagctctgttcaggcagaagatctggctgtctattactgccagcaacattatagcatcccgttcacgttcggtgctgggaccaagctggagctgaaa

SEQ ID NO: 4 - protein, 107 aa, Artificial sequence
  Design 1 light chain variable domain sequence
DVVMTQTHKFMSTSVGDRVSITCKASQDVSTAVAWYQQKSGQSPKLLIHSASYRHTGVPDRFTGSGSGTDFTFTISSVQAEDLAVYYCQQHYSIPFTFGAGTKLELK

SEQ ID NO: 5 - DNA, 321 bp, Artificial sequence
  Design 2 light chain variable domain sequence
gatgtggttatgacccaaacgcataaattcatgagcacctctgtgggcgatcgtgttagtattacgtgtaaggcatcacaggacgtctcgaccgcagtggcctggtatcagcaaaaaagtggtcaatccccgaagctgctgatccacagtgcctcctatcgtcccaccggcgttccggatcgctttaccggctcaggttcgggcacggactttaccttcacgattagctctgttcaggcagaagatctggctgtctattactgccagcaacattatagcatcccgttcacgttcggtgctgggaccaagctggagctgaaa

SEQ ID NO: 6 - protein, 107 aa, Artificial sequence
  Design 2 light chain variable domain sequence
DVVMTQTHKFMSTSVGDRVSITCKASQDVSTAVAWYQQKSGQSPKLLIHSASYRPTGVPDRFTGSGSGTDFTFTISSVQAEDLAVYYCQQHYSIPFTFGAGTKLELK

SEQ ID NO: 7 - DNA, 321 bp, Artificial sequence
  Design 3 light chain variable domain sequence
gatgtggttatgacccaaacgcataaattcatgagcacctctgtgggcgatcgtgttagtattacgtgtaaggcatcacaggacgtctcgaccgcagtggcctggtatcagcaaaaaagtggtcaagccccgaagctgctgatccacagtgcctcctatcgtcccaccggcgttccggatcgctttaccggctcaggttcgggcacggactttaccttcacgattagctctgttcaggcagaagatctggctgtctattactgccagcaacattatagcatcccgctcacgttcggtgctgggaccaagctggagctgaaa

SEQ ID NO: 8 - protein, 107 aa, Artificial sequence
  Design 3 light chain variable domain sequence
DVVMTQTHKFMSTSVGDRVSITCKASQDVSTAVAWYQQKSGQAPKLLIHSASYRPTGVPDRFTGSGSGTDFTFTISSVQAEDLAVYYCQQHYSIPLTFGAGTKLELK

SEQ ID NO: 9 - DNA, 321 bp, Artificial sequence
  Design 4 light chain variable domain sequence
gatgtggttatgacccaaacgcataaattcatgagcacctctgtgggcgatcgtgttagtattacgtgtaaggcatcacaggacgtctcgaccgcagtggcctggtatcagcaaaaaagtggtcaacccccgaagctgctgatccacagtgcctcctatcgtcacaccggcgttccggatcgctttaccggctcaggttcgggcacggactttaccttcacgattagctctgttcaggcagaagatctggctgtctattactgccagcaacattatagcatcccgctcacgttcggtgctgggaccaagctggagctgaaa

SEQ ID NO: 10 - protein, 107 aa, Artificial sequence
  Design 4 light chain variable domain sequence
DVVMTQTHKFMSTSVGDRVSITCKASQDVSTAVAWYQQKSGQPPKLLIHSASYRHTGVPDRFTGSGSGTDFTFTISSVQAEDLAVYYCQQHYSIPLTFGAGTKLELK

SEQ ID NO: 11 - DNA, 321 bp, Artificial sequence
  Design 5 light chain variable domain sequence
gatgtggttatgacccaaacgcataaattcatgagcacctctgtgggcgatcgtgttagtattacgtgtaaggcatcacaggacgtctcgaccgcagtggcctggtatcagcaaaaaagtggtcaacccccgaagctgctgatccacagtgcctcctatcgtcccaccggcgttccggatcgctttaccggctcaggttcgggcacggactttaccttcacgattagctctgttcaggcagaagatctggctgtctattactgccagcaacattatagcatcccgttcacgttcggtgctgggaccaagctggagctgaaa

SEQ ID NO: 12 - protein, 107 aa, Artificial sequence
  Design 5 light chain variable domain sequence
DVVMTQTHKFMSTSVGDRVSITCKASQDVSTAVAWYQQKSGQPPKLLIHSASYRPTGVPDRFTGSGSGTDFTFTISSVQAEDLAVYYCQQHYSIPFTFGAGTKLELK

SEQ ID NO: 13 - DNA, 321 bp, Artificial sequence; identical to SEQ ID NO: 11
  Design 6 light chain variable domain sequence

SEQ ID NO: 14 - protein, 107 aa, Artificial sequence; identical to SEQ ID NO: 12
  Design 6 light chain variable domain sequence

SEQ ID NO: 15 - DNA, 321 bp, Artificial sequence; identical to SEQ ID NO: 11
  Design 7 light chain variable domain sequence

SEQ ID NO: 16 - protein, 107 aa, Artificial sequence; identical to SEQ ID NO: 12
  Design 7 light chain variable domain sequence

SEQ ID NO: 17 - DNA, 321 bp, Artificial sequence; identical to SEQ ID NO: 11
  Design 8 light chain variable domain sequence

SEQ ID NO: 18 - protein, 107 aa, Artificial sequence; identical to SEQ ID NO: 12
  Design 8 light chain variable domain sequence

SEQ ID NO: 19 - DNA, 321 bp, Artificial sequence; identical to SEQ ID NO: 11
  Design 9 light chain variable domain sequence

SEQ ID NO: 20 - protein, 107 aa, Artificial sequence; identical to SEQ ID NO: 12
  Design 9 light chain variable domain sequence

SEQ ID NO: 21 - DNA, 321 bp, Artificial sequence; identical to SEQ ID NO: 11
  Design 10 light chain variable domain sequence

SEQ ID NO: 22 - protein, 107 aa, Artificial sequence; identical to SEQ ID NO: 12
  Design 10 light chain variable domain sequence

SEQ ID NO: 23 - DNA, 321 bp, Artificial sequence
  Design 11 light chain variable domain sequence
gatgtggttatgacccaaacgcataaattcatgagcacctctgtgggcgatcgtgttagtattacgtgtaaggcatcacaggacgtctcgaccgcagtggcctggtatcagcaaaaaagtggtcaacccccgaagctgctgatccacagtgcctcctatcgtcccaccggcgttccggatcgctttaccggctcaggttcgggcacggactttaccttcacgattagctctgttcaggcagaagatctggctgtctattactgccatcaacattatagcatcccgctcacgttcggtgctgggaccaagctggagctgaaa

SEQ ID NO: 24 - protein, 107 aa, Artificial sequence
  Design 11 light chain variable domain sequence
DVVMTQTHKFMSTSVGDRVSITCKASQDVSTAVAWYQQKSGQPPKLLIHSASYRPTGVPDRFTGSGSGTDFTFTISSVQAEDLAVYYCHQHYSIPLTFGAGTKLELK

SEQ ID NO: 25 - DNA, 321 bp, Artificial sequence; identical to SEQ ID NO: 23
  Design 12 light chain variable domain sequence

SEQ ID NO: 26 - protein, 107 aa, Artificial sequence; identical to SEQ ID NO: 24
  Design 12 light chain variable domain sequence

SEQ ID NO: 27 - DNA, 321 bp, Artificial sequence
  Design 13 light chain variable domain sequence
gatgtggttatgacccaaacgcataaattcatgagcacctctgtgggcgatcgtgttagtattacgtgtaaggcatcacaggacgtctcgaccgcagtggcctggtatcagcaaaaaagtggtcaacccccgaagctgctgatccacagtgcctcctatcgtcccaccggcgttccggatcgctttaccggctcaggttcgggcacggactttaccttcacgattagctctgttcaggcagaagatctggctgtctattactgccatcaacattatagcatcccgttcacgttcggtgctgggaccaagctggagctgaaa

SEQ ID NO: 28 - protein, 107 aa, Artificial sequence
  Design 13 light chain variable domain sequence
DVVMTQTHKFMSTSVGDRVSITCKASQDVSTAVAWYQQKSGQPPKLLIHSASYRPTGVPDRFTGSGSGTDFTFTISSVQAEDLAVYYCHQHYSIPFTFGAGTKLELK

SEQ ID NO: 29 - DNA, 321 bp, Artificial sequence; identical to SEQ ID NO: 27
  Design 14 light chain variable domain sequence

SEQ ID NO: 30 - protein, 107 aa, Artificial sequence; identical to SEQ ID NO: 28
  Design 14 light chain variable domain sequence

SEQ ID NO: 31 - DNA, 321 bp, Artificial sequence; identical to SEQ ID NO: 27
  Design 15 light chain variable domain sequence

SEQ ID NO: 32 - protein, 107 aa, Artificial sequence; identical to SEQ ID NO: 28
  Design 15 light chain variable domain sequence

SEQ ID NO: 33 - DNA, 321 bp, Artificial sequence
  Design 16 light chain variable domain sequence
gatgtggttatgacccaaacgcataaattcatgagcacctctgtgggcgatcgtgttagtattacgtgtaaggcatcacaggacgtctcgaccgcagtggcctggtatcagcaaaaaagtggtcaacccccgaagctgctgatccacagtgcctcctatcgtgtcaccggcgttccggatcgctttaccggctcaggttcgggcacggactttaccttcacgattagctctgttcaggcagaagatctggctgtctattactgccagcaacattatagcatcccgttcacgttcggtgctgggaccaagctggagctgaaa

SEQ ID NO: 34 - protein, 107 aa, Artificial sequence
  Design 16 light chain variable domain sequence
DVVMTQTHKFMSTSVGDRVSITCKASQDVSTAVAWYQQKSGQPPKLLIHSASYRVTGVPDRFTGSGSGTDFTFTISSVQAEDLAVYYCQQHYSIPFTFGAGTKLELK

SEQ ID NO: 35 - DNA, 321 bp, Artificial sequence; identical to SEQ ID NO: 33
  Design 17 light chain variable domain sequence

SEQ ID NO: 36 - protein, 107 aa, Artificial sequence; identical to SEQ ID NO: 34
  Design 17 light chain variable domain sequence

SEQ ID NO: 37 - DNA, 321 bp, Artificial sequence; identical to SEQ ID NO: 33
  Design 18 light chain variable domain sequence

SEQ ID NO: 38 - protein, 107 aa, Artificial sequence; identical to SEQ ID NO: 34
  Design 18 light chain variable domain sequence

SEQ ID NO: 39 - DNA, 321 bp, Artificial sequence
  Design 19 light chain variable domain sequence
gatgtggttatgacccaaacgcataaattcatgagcacctctgtgggcgatcgtgttagtattacgtgtaaggcatcacaggacgtctcgaccgcagtggcctggtatcagcaaaaaagtggtcaacccccgaagctgctgatccacagtgcctcctatcgtgtcaccggcgttccggatcgctttaccggctcaggttcgggcacggactttaccttcacgattagctctgttcaggcagaagatctggctgtctattactgccatcaacattatagcatcccgctcacgttcggtgctgggaccaagctggagctgaaa

SEQ ID NO: 40 - protein, 107 aa, Artificial sequence
  Design 19 light chain variable domain sequence
DVVMTQTHKFMSTSVGDRVSITCKASQDVSTAVAWYQQKSGQPPKLLIHSASYRVTGVPDRFTGSGSGTDFTFTISSVQAEDLAVYYCHQHYSIPLTFGAGTKLELK

SEQ ID NO: 41 - DNA, 321 bp, Artificial sequence
  Design 20 light chain variable domain sequence
gatgtggttatgacccaaacgcataaattcatgagcacctctgtgggcgatcgtgttagtattacgtgtaaggcatcacaggacgtctcgaccgcagtggcctggtatcagcaaaaaagtggtcaacccccgaagctgctgatccacagtgcctcctatcgtgtcaccggcgttccggatcgctttaccggctcaggttcgggcacggactttaccttcacgattagctctgttcaggcagaagatctggctgtctattactgccatcaacattatagcatcccgttcacgttcggtgctgggaccaagctggagctgaaa

SEQ ID NO: 42 - protein, 107 aa, Artificial sequence
  Design 20 light chain variable domain sequence
DVVMTQTHKFMSTSVGDRVSITCKASQDVSTAVAWYQQKSGQPPKLLIHSASYRVTGVPDRFTGSGSGTDFTFTISSVQAEDLAVYYCHQHYSIPFTFGAGTKLELK

SEQ ID NO: 43 - DNA, 354 bp, Artificial sequence
  Design 1 heavy chain variable domain sequence
caagtccaactgaagcaaagcggtccgggtctggtcgcgccgtcccaaagcctgtcgattacctgcaccgtctcgggttttagcctgaccggctatggtgttaactgggtccgtcagtcgccgggcaaagctctggaatggctgggtatgatttggggcgatggtcgtacggactacaaatcagcactgaagagccgcctgtctatcaccaaagataacagtaagtcccaggtgtttctgaaaatgaattccctgcaaaccgatgacacggcgcgctattattgcgcctcagactattacggctcaggtgcgttggcatactggggccagggtaccctggtcacggtgtctgct

SEQ ID NO: 44 - protein, 118 aa, Artificial sequence
  Design 1 heavy chain variable domain sequence
QVQLKQSGPGLVAPSQSLSITCTVSGFSLTGYGVNWVRQSPGKALEWLGMIWGDGRTDYKSALKSRLSITKDNSKSQVFLKMNSLQTDDTARYYCASDYYGSGALAYWGQGTLVTVSA

SEQ ID NO: 45 - DNA, 354 bp, Artificial sequence
  Design 2 heavy chain variable domain sequence
caagtccaactgaagcaaagcggtccgggtctggtcgcgccgtcccaaagcctgtcgattacctgcaccgtctcgggttttagcctgaccggctatggtgttaactgggtccgtcagtcgccgggcaaaggtctggaatggctgggtatgatttggggcgatggtcgtacggagtacaaatcagcactgaagagccgcctgtctatcaccaaagataacagtaagtcccaggtgtttctgaaaatgaattccctgcaaaccgatgacacggcgcgctatttttgcgcctcagactattacggctcaggtgcgttcgaatactggggccagggtaccctggtcacggtgtctgct

SEQ ID NO: 46 - protein, 118 aa, Artificial sequence
  Design 2 heavy chain variable domain sequence
QVQLKQSGPGLVAPSQSLSITCTVSGFSLTGYGVNWVRQSPGKGLEWLGMIWGDGRTEYKSALKSRLSITKDNSKSQVFLKMNSLQTDDTARYFCASDYYGSGAFEYWGQGTLVTVSA

SEQ ID NO: 47 - DNA, 354 bp, Artificial sequence
  Design 3 heavy chain variable domain sequence
caagtccaactgaagcaaagcggtccgggtctggtcgcgccgtcccaaagcctgtcgattacctgcaccgtctcgggttttagcctgaccggctatggtgttaactgggtccgtcagtcgccgggcaaaggtctggaatggctgggtatgatttggggcgatggtcgtacggagtacaaatcagcactgaagagccgcctgtctatcaccaaagataacagtaagtcccaggtgtttctgaaaatgaattccctgcaaaccgatgacacggcgcgctattattgcgcctcagactattacggctcaggtgcgttggaatactggggccagggtaccctggtcacggtgtctgct

SEQ ID NO: 48 - protein, 118 aa, Artificial sequence
  Design 3 heavy chain variable domain sequence
QVQLKQSGPGLVAPSQSLSITCTVSGFSLTGYGVNWVRQSPGKGLEWLGMIWGDGRTEYKSALKSRLSITKDNSKSQVFLKMNSLQTDDTARYYCASDYYGSGALEYWGQGTLVTVSA

SEQ ID NO: 49 - DNA, 354 bp, Artificial sequence
  Design 4 heavy chain variable domain sequence
caagtccaactgaagcaaagcggtccgggtctggtcgcgccgtcccaaagcctgtcgattacctgcaccgtctcgggttttagcctgaccggctatggtgttaactgggtccgtcagtcgccgggcaaaggtctggaatggctgggtatgatttggggcgatggtcgtacggagtacaaatcagcactgaagagccgcctgtctatcaccaaagataacagtaagtcccaggtgtttctgaaaatgaattccctgcaaaccgatgacacggcgcgctattattgcgcctcagactattacggctcaggttcgttcccatactggggccagggtaccctggtcacggtgtctgct

SEQ ID NO: 50 - protein, 118 aa, Artificial sequence
  Design 4 heavy chain variable domain sequence
QVQLKQSGPGLVAPSQSLSITCTVSGFSLTGYGVNWVRQSPGKGLEWLGMIWGDGRTEYKSALKSRLSITKDNSKSQVFLKMNSLQTDDTARYYCASDYYGSGSFPYWGQGTLVTVSA

SEQ ID NO: 51 - DNA, 354 bp, Artificial sequence
  Design 5 heavy chain variable domain sequence
caagtccaactgaagcaaagcggtccgggtctggtcgcgccgtcccaaagcctgtcgattacctgcaccgtctcgggttttagcctgaccggctatggtgttaactgggtccgtcagtcgccgggcaaaggtctggaatggctgggtatgatttggggcgatggtcgtacggactacaaatcagcactgaagagccgcctgtctatcaccaaagataacagtaagtcccaggtgtttctgaaaatgaattccctgcaaaccgatgacacggcgcgctatttttgcgcctcagactattacggctcaggttcgttcaattactggggccagggtaccctggtcacggtgtctgct

SEQ ID NO: 52 - protein, 118 aa, Artificial sequence
  Design 5 heavy chain variable domain sequence
QVQLKQSGPGLVAPSQSLSITCTVSGFSLTGYGVNWVRQSPGKGLEWLGMIWGDGRTDYKSALKSRLSITKDNSKSQVFLKMNSLQTDDTARYFCASDYYGSGSFNYWGQGTLVTVSA

SEQ ID NO: 53 - DNA, 354 bp, Artificial sequence
  Design 6 heavy chain variable domain sequence
caagtccaactgaagcaaagcggtccgggtctggtcgcgccgtcccaaagcctgtcgattacctgcaccgtctcgggttttagcctgaccggctatggtgttaactgggtccgtcagtcgccgggcaaaggtctggaatggctgggtatgatttggggcgatggtcgtacggactacaaatcagcactgaagagccgcctgtctatcaccaaagataacagtaagtcccaggtgtttctgaaaatgaattccctgcaaaccgatgacacggcgcgctatttttgcgcctcagactattacggctcaggtgcgttggaatactggggccagggtaccctggtcacggtgtctgct

SEQ ID NO: 54 - protein, 118 aa, Artificial sequence
  Design 6 heavy chain variable domain sequence
QVQLKQSGPGLVAPSQSLSITCTVSGFSLTGYGVNWVRQSPGKGLEWLGMIWGDGRTDYKSALKSRLSITKDNSKSQVFLKMNSLQTDDTARYFCASDYYGSGALEYWGQGTLVTVSA

SEQ ID NO: 55 - DNA, 354 bp, Artificial sequence
  Design 7 heavy chain variable domain sequence
caagtccaactgaagcaaagcggtccgggtctggtcgcgccgtcccaaagcctgtcgattacctgcaccgtctcgggttttagcctgaccggctatggtgttaactgggtccgtcagtcgccgggcaaaggtctggaatggctgggtatgatttggggcgatggtcgtacggagtacaaatcagcactgaagagccgcctgtctatcaccaaagataacagtaagtcccaggtgtttctgaaaatgaattccctgcaaaccgatgacacggcgcgctattattgcgcctcagactattacggctcaggttcgttagaatactggggccagggtaccctggtcacggtgtctgct

SEQ ID NO: 56 - protein, 118 aa, Artificial sequence
  Design 7 heavy chain variable domain sequence
QVQLKQSGPGLVAPSQSLSITCTVSGFSLTGYGVNWVRQSPGKGLEWLGMIWGDGRTEYKSALKSRLSITKDNSKSQVFLKMNSLQTDDTARYYCASDYYGSGSLEYWGQGTLVTVSA

SEQ ID NO: 57 - DNA, 354 bp, Artificial sequence
  Design 8 heavy chain variable domain sequence
caagtccaactgaagcaaagcggtccgggtctggtcgcgccgtcccaaagcctgtcgattacctgcaccgtctcgggttttagcctgaccggctatggtgttaactgggtccgtcagtcgccgggcaaagctctggaatggctgggtatgatttggggcgatggtcgtacggactacaaatcagcactgaagagccgcctgtctatcaccaaagataacagtaagtcccaggtgtttctgaaaatgaattccctgcaaaccgatgacacggcgcgctatttttgcgcctcagactattacggctcaggttcgtaccaatactggggccagggtaccctggtcacggtgtctgct

SEQ ID NO: 58 - protein, 118 aa, Artificial sequence
  Design 8 heavy chain variable domain sequence
QVQLKQSGPGLVAPSQSLSITCTVSGFSLTGYGVNWVRQSPGKALEWLGMIWGDGRTDYKSALKSRLSITKDNSKSQVFLKMNSLQTDDTARYFCASDYYGSGSYQYWGQGTLVTVSA

SEQ ID NO: 59 - DNA, 354 bp, Artificial sequence
  Design 9 heavy chain variable domain sequence
caagtccaactgaagcaaagcggtccgggtctggtcgcgccgtcccaaagcctgtcgattacctgcaccgtctcgggttttagcctgaccggctatggtgttaactgggtccgtcagtcgccgggcaaagctctggaatggctgggtatgatttggggcgatggtcgtacggactacaaatcagcactgaagagccgcctgtctatcaccaaagataacagtaagtcccaggtgtttctgaaaatgaattccctgcaaaccgatgacacggcgcgctattattgcgcctcagactattacggctcaggtgcgttgcaatactggggccagggtaccctggtcacggtgtctgct

SEQ ID NO: 60 - protein, 118 aa, Artificial sequence
  Design 9 heavy chain variable domain sequence
QVQLKQSGPGLVAPSQSLSITCTVSGFSLTGYGVNWVRQSPGKALEWLGMIWGDGRTDYKSALKSRLSITKDNSKSQVFLKMNSLQTDDTARYYCASDYYGSGALQYWGQGTLVTVSA

SEQ ID NO: 61 - DNA, 354 bp, Artificial sequence
  Design 10 heavy chain variable domain sequence
caagtccaactgaagcaaagcggtccgggtctggtcgcgccgtcccaaagcctgtcgattacctgcaccgtctcgggttttagcctgaccggctatggtgttaactgggtccgtcagtcgccgggcaaagctctggaatggctgggtatgatttggggcgatggtcgtacggagtacaaatcagcactgaagagccgcctgtctatcaccaaagataacagtaagtcccaggtgtttctgaaaatgaattccctgcaaaccgatgacacggcgcgctatttttgcgcctcagactattacggctcaggttcgttggcatactggggccagggtaccctggtcacggtgtctgct

SEQ ID NO: 62 - protein, 118 aa, Artificial sequence
  Design 10 heavy chain variable domain sequence
QVQLKQSGPGLVAPSQSLSITCTVSGFSLTGYGVNWVRQSPGKALEWLGMIWGDGRTEYKSALKSRLSITKDNSKSQVFLKMNSLQTDDTARYFCASDYYGSGSLAYWGQGTLVTVSA

SEQ ID NO: 63 - DNA, 354 bp, Artificial sequence
  Design 11 heavy chain variable domain sequence
caagtccaactgaagcaaagcggtccgggtctggtcgcgccgtcccaaagcctgtcgattacctgcaccgtctcgggttttagcctgaccggctatggtgttaactgggtccgtcagtcgccgggcaaaggtctggaatggctgggtatgatttggggcgatggtcgtacggactacaaatcagcactgaagagccgcctgtctatcaccaaagataacagtaagtcccaggtgtttctgaaaatgaattccctgcaaaccgatgacacggcgcgctatttttgcgcctcagactattacggctcaggtgcgttccaatactggggccagggtaccctggtcacggtgtctgct

SEQ ID NO: 64 - protein, 118 aa, Artificial sequence
  Design 11 heavy chain variable domain sequence
QVQLKQSGPGLVAPSQSLSITCTVSGFSLTGYGVNWVRQSPGKGLEWLGMIWGDGRTDYKSALKSRLSITKDNSKSQVFLKMNSLQTDDTARYFCASDYYGSGAFQYWGQGTLVTVSA

SEQ ID NO: 65 - DNA, 354 bp, Artificial sequence
  Design 12 heavy chain variable domain sequence
caagtccaactgaagcaaagcggtccgggtctggtcgcgccgtcccaaagcctgtcgattacctgcaccgtctcgggttttagcctgaccggctatggtgttaactgggtccgtcagtcgccgggcaaagctctggaatggctgggtatgatttggggcgatggtcgtacggactacaaatcagcactgaagagccgcctgtctatcaccaaagataacagtaagtcccaggtgtttctgaaaatgaattccctgcaaaccgatgacacggcgcgctattattgcgcctcagactattacggctcaggttcgttccaatactggggccagggtaccctggtcacggtgtctgct

SEQ ID NO: 66 - protein, 118 aa, Artificial sequence
  Design 12 heavy chain variable domain sequence
QVQLKQSGPGLVAPSQSLSITCTVSGFSLTGYGVNWVRQSPGKALEWLGMIWGDGRTDYKSALKSRLSITKDNSKSQVFLKMNSLQTDDTARYYCASDYYGSGSFQYWGQGTLVTVSA

SEQ ID NO: 67 - DNA, 354 bp, Artificial sequence
  Design 13 heavy chain variable domain sequence
caagtccaactgaagcaaagcggtccgggtctggtcgcgccgtcccaaagcctgtcgattacctgcaccgtctcgggttttagcctgaccggctatggtgttaactgggtccgtcagtcgccgggcaaaggtctggaatggctgggtatgatttggggcgatggtcgtacggactacaaatcagcactgaagagccgcctgtctatcaccaaagataacagtaagtcccaggtgtttctgaaaatgaattccctgcaaaccgatgacacggcgcgctatttttgcgcctcagactattacggctcaggttcgttggcatactggggccagggtaccctggtcacggtgtctgct

SEQ ID NO: 68 - protein, 118 aa, Artificial sequence
  Design 13 heavy chain variable domain sequence
QVQLKQSGPGLVAPSQSLSITCTVSGFSLTGYGVNWVRQSPGKGLEWLGMIWGDGRTDYKSALKSRLSITKDNSKSQVFLKMNSLQTDDTARYFCASDYYGSGSLAYWGQGTLVTVSA

SEQ ID NO: 69 - DNA, 354 bp, Artificial sequence
  Design 14 heavy chain variable domain sequence
caagtccaactgaagcaaagcggtccgggtctggtcgcgccgtcccaaagcctgtcgattacctgcaccgtctcgggttttagcctgaccggctatggtgttaactgggtccgtcagtcgccgggcaaaggtctggaatggctgggtatgatttggggcgatggtcgtacggactacaaatcagcactgaagagccgcctgtctatcaccaaagataacagtaagtcccaggtgtttctgaaaatgaattccctgcaaaccgatgacacggcgcgctattattgcgcctcagactattacggctcaggtgcgtacgaatactggggccagggtaccctggtcacggtgtctgct

SEQ ID NO: 70 - protein, 118 aa, Artificial sequence
  Design 14 heavy chain variable domain sequence
QVQLKQSGPGLVAPSQSLSITCTVSGFSLTGYGVNWVRQSPGKGLEWLGMIWGDGRTDYKSALKSRLSITKDNSKSQVFLKMNSLQTDDTARYYCASDYYGSGAYEYWGQGTLVTVSA

SEQ ID NO: 71 - DNA, 354 bp, Artificial sequence
  Design 15 heavy chain variable domain sequence
caagtccaactgaagcaaagcggtccgggtctggtcgcgccgtcccaaagcctgtcgattacctgcaccgtctcgggttttagcctgaccggctatggtgttaactgggtccgtcagtcgccgggcaaaggtctggaatggctgggtatgatttggggcgatggtcgtacggagtacaaatcagcactgaagagccgcctgtctatcaccaaagataacagtaagtcccaggtgtttctgaaaatgaattccctgcaaaccgatgacacggcgcgctatttttgcgcctcagactattacggctcaggttcgtaccaatactggggccagggtaccctggtcacggtgtctgct

SEQ ID NO: 72 - protein, 118 aa, Artificial sequence
  Design 15 heavy chain variable domain sequence
QVQLKQSGPGLVAPSQSLSITCTVSGFSLTGYGVNWVRQSPGKGLEWLGMIWGDGRTEYKSALKSRLSITKDNSKSQVFLKMNSLQTDDTARYFCASDYYGSGSYQYWGQGTLVTVSA

SEQ ID NO: 73 - DNA, 354 bp, Artificial sequence
  Design 16 heavy chain variable domain sequence
caagtccaactgaagcaaagcggtccgggtctggtcgcgccgtcccaaagcctgtcgattacctgcaccgtctcgggttttagcctgaccggctatggtgttaactgggtccgtcagtcgccgggcaaaggtttcgaatggctgggtatgatttggggcgatggtcgtacggactacaaatcagcactgaagagccgcctgtctatcaccaaagataacagtaagtcccaggtgtttctgaaaatgaattccctgcaaaccgatgacacggcgcgctatttttgcgcctcagactattacggctcaggttcgtaccaatactggggccagggtaccctggtcacggtgtctgct

SEQ ID NO: 74 - protein, 118 aa, Artificial sequence
  Design 16 heavy chain variable domain sequence
QVQLKQSGPGLVAPSQSLSITCTVSGFSLTGYGVNWVRQSPGKGFEWLGMIWGDGRTDYKSALKSRLSITKDNSKSQVFLKMNSLQTDDTARYFCASDYYGSGSYQYWGQGTLVTVSA

SEQ ID NO: 75 - DNA, 354 bp, Artificial sequence
  Design 17 heavy chain variable domain sequence
caagtccaactgaagcaaagcggtccgggtctggtcgcgccgtcccaaagcctgtcgattacctgcaccgtctcgggttttagcctgaccggctatggtgttaactgggtccgtcagtcgccgggcaaagctctggaatggctgggtatgatttggggcgatggtcgtacggactacaaatcagcactgaagagccgcctgtctatcaccaaagataacagtaagtcccaggtgtttctgaaaatgaattccctgcaaaccgatgacacggcgcgctatttttgcgcctcagactattacggctcaggtgcgttggcatactggggccagggtaccctggtcacggtgtctgct

SEQ ID NO: 76 - protein, 118 aa, Artificial sequence
  Design 17 heavy chain variable domain sequence
QVQLKQSGPGLVAPSQSLSITCTVSGFSLTGYGVNWVRQSPGKALEWLGMIWGDGRTDYKSALKSRLSITKDNSKSQVFLKMNSLQTDDTARYFCASDYYGSGALAYWGQGTLVTVSA

SEQ ID NO: 77 - DNA, 354 bp, Artificial sequence
  Design 18 heavy chain variable domain sequence
caagtccaactgaagcaaagcggtccgggtctggtcgcgccgtcccaaagcctgtcgattacctgcaccgtctcgggttttagcctgaccggctatggtgttaactggttccgtcagtcgccgggcaaaggtctggaatggctgggtatgatttggggcgatggtcgtacggactacaaatcagcactgaagagccgcctgtctatcaccaaagataacagtaagtcccaggtgtttctgaaaatgaattccctgcaaaccgatgacacggcgcgctatttttgcgcctcagactattacggctcaggttcgttcgcatactggggccagggtaccctggtcacggtgtctgct

SEQ ID NO: 78 - protein, 118 aa, Artificial sequence
  Design 18 heavy chain variable domain sequence
QVQLKQSGPGLVAPSQSLSITCTVSGFSLTGYGVNWFRQSPGKGLEWLGMIWGDGRTDYKSALKSRLSITKDNSKSQVFLKMNSLQTDDTARYFCASDYYGSGSFAYWGQGTLVTVSA

SEQ ID NO: 79 - DNA, 354 bp, Artificial sequence; identical to SEQ ID NO: 61
  Design 19 heavy chain variable domain sequence

SEQ ID NO: 80 - protein, 118 aa, Artificial sequence
  Design 19 heavy chain variable domain sequence
QVQLKQSGPGLVAPSQSLSITCTVSGFSLTGYGVNWVRQSPGKALEWLGMIWGDGRTEYKSALKSRLSITKDNSKSQVFLKMNSLQTDDTARYYCASDYYGSGALAYWGQGTLVTVSA

SEQ ID NO: 81 - DNA, 354 bp, Artificial sequence; identical to SEQ ID NO: 43
  Design 20 heavy chain variable domain sequence

SEQ ID NO: 82 - protein, 118 aa, Artificial sequence
  Design 20 heavy chain variable domain sequence
QVQLKQSGPGLVAPSQSLSITCTVSGFSLTGYGVNWVRQSPGKALEWLGMIWGDGRTDYKSALKSRLSITKDNSKSQVFLKMNSLQTDDTARYFCASDYYGSGSYAYWGQGTLVTVSA

SEQ ID NO: 83 - protein, 8 aa, Artificial sequence
  MAb 492.2 complementarity-determining region (CDR) H1
GFSLTGYG

SEQ ID NO: 84 - protein, 7 aa, Artificial sequence
  MAb 492.2 complementarity-determining region (CDR) H2
IWGDGRT

SEQ ID NO: 85 - protein, 12 aa, Artificial sequence
  MAb 492.2 complementarity-determining region (CDR) H3
ASDYYGSGSFAY

SEQ ID NO: 86 - protein, 6 aa, Artificial sequence
  MAb 492.2 complementarity-determining region (CDR) L1
QDVSTA

SEQ ID NO: 87 - protein, 3 aa, Artificial sequence
  MAb 492.2 complementarity-determining region (CDR) L2
SAS

SEQ ID NO: 88 - protein, 9 aa, Artificial sequence
  MAb 492.2 complementarity-determining region (CDR) L3
QQHYSIPLT

SEQ ID NO: 89 - DNA, 1401 bp, Artificial sequence
  Mab 492.1 (Chimeric antibodies human constant regions) heavy chain nucleic acid sequence
atgggatggtcatgtatcatcctttttctagtagcaactgcaaccggtgtacattgccaagtccaactgaagcaaagcggtccgggtctggtcgcgccgtcccaaagcctgtcgattacctgcaccgtctcgggttttagcctgaccggctatggtgttaactgggtccgtcagtcgccgggcaaaggtctggaatggctgggtatgatttggggcgatggtcgtacggactacaaatcagcactgaagagccgcctgtctatcaccaaagataacagtaagtcccaggtgtttctgaaaatgaattccctgcaaaccgatgacacggcgcgctatttttgcgcctcagactattacggctcaggttcgttcgcatactggggccagggtaccctggtcacggtgtctgcttcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgctgcatgaggctctgcacagccactacacgcagaagagcctctccctgtccccgggtaaatga

SEQ ID NO: 90 - DNA, 702 bp, Artificial sequence
  Mab 492.1 (Chimeric antibodies human constant regions) light chain nucleic acid sequence
atgggatggtcatgtatcatcctttttctagtagcaactgcaaccggtgtacattctgatgtggttatgacccaaacgcataaattcatgagcacctctgtgggcgatcgtgttagtattacgtgtaaggcatcacaggacgtctcgaccgcagtggcctggtatcagcaaaaaagtggtcaatccccgaagctgctgatccacagtgcctcctatcgttacaccggcgttccggatcgctttaccggctcaggttcgggcacggactttaccttcacgattagctctgttcaggcagaagatctggctgtctattactgccagcaacattatagcatcccgctcacgttcggtgctgggaccaagctggagctgaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 91 - DNA, 321 bp, Artificial sequence
  Design 21 light chain variable domain sequence
gatgtggttatgacccaaacgcataaattcatgagcacctctgtgggcgatcgtgttagtattacgtgtaaggcatcacaggacgtctcgaccgcagtggcctggtatcagcaaaaaagtggtcaacccccgaagctgctgatccacagtgcctcctatcgttacaccggcgttccggatcgctttaccggctcaggttcgggcacggactttaccttcacgattagctctgttcaggcagaagatctggctgtctattactgccagcaacattatagcatcccgctcacgttcggtgctgggaccaagctggagctgaaa

SEQ ID NO: 92 - protein, 107 aa, Artificial sequence
  Design 21 light chain variable domain sequence
DVVMTQTHKFMSTSVGDRVSITCKASQDVSTAVAWYQQKSGQPPKLLIHSASYRYTGVPDRFTGSGSGTDFTFTISSVQAEDLAVYYCQQHYSIPLTFGAGTKLELK

SEQ ID NO: 93 - DNA, 321 bp, Artificial sequence
  Design 22 light chain variable domain sequence
gatgtggttatgacccaaacgcataaattcatgagcacctctgtgggcgatcgtgttagtattacgtgtaaggcatcacaggacgtctcgaccgcagtggcctggtatcagcaaaaaagtggtcaatccccgaagctgctgatccacagtgcctcctatcgtcccaccggcgttccggatcgctttaccggctcaggttcgggcacggactttaccttcacgattagctctgttcaggcagaagatctggctgtctattactgccagcaacattatagcatcccgctcacgttcggtgctgggaccaagctggagctgaaa

SEQ ID NO: 94 - protein, 107 aa, Artificial sequence
  Design 22 light chain variable domain sequence
DVVMTQTHKFMSTSVGDRVSITCKASQDVSTAVAWYQQKSGQSPKLLIHSASYRPTGVPDRFTGSGSGTDFTFTISSVQAEDLAVYYCQQHYSIPLTFGAGTKLELK

SEQ ID NO: 95 - DNA, 321 bp, Artificial sequence
  Design 23 light chain variable domain sequence
gatgtggttatgacccaaacgcataaattcatgagcacctctgtgggcgatcgtgttagtattacgtgtaaggcatcacaggacgtctcgaccgcagtggcctggtatcagcaaaaaagtggtcaacccccgaagctgctgatccacagtgcctcctatcgtcccaccggcgttccggatcgctttaccggctcaggttcgggcacggactttaccttcacgattagctctgttcaggcagaagatctggctgtctattactgccagcaacattatagcatcccgctcacgttcggtgctgggaccaagctggagctgaaa

SEQ ID NO: 96 - protein, 107 aa, Artificial sequence
  Design 23 light chain variable domain sequence
DVVMTQTHKFMSTSVGDRVSITCKASQDVSTAVAWYQQKSGQPPKLLIHSASYRPTGVPDRFTGSGSGTDFTFTISSVQAEDLAVYYCQQHYSIPLTFGAGTKLELK

SEQ ID NO: 97 - protein, 12 aa, Artificial sequence
  complementarity-determining region (CDR) H3
  misc_feature (9)..(11): Xaa can be any naturally occurring amino acid
ASDYYGSGXXXY

SEQ ID NO: 98 - protein, 9 aa, Artificial sequence
  complementarity-determining region (CDR) L3
  misc_feature (8)..(8): Xaa can be any naturally occurring amino acid
QQHYSIPXT

SEQ ID NO: 99 - protein, 12 aa, Artificial sequence
  complementarity-determining region (CDR) H3
ASDYYGSGALEY

SEQ ID NO: 100 - protein, 12 aa, Artificial sequence
  complementarity-determining region (CDR) H3
ASDYYGSGALAY

SEQ ID NO: 101 - protein, 12 aa, Artificial sequence
  complementarity-determining region (CDR) H3
ASDYYGSGAFEY

SEQ ID NO: 102 - protein, 12 aa, Artificial sequence
  complementarity-determining region (CDR) H3
ASDYYGSGALEY

SEQ ID NO: 103 - protein, 12 aa, Artificial sequence
  complementarity-determining region (CDR) H3
ASDYYGSGSFPY

SEQ ID NO: 104 - protein, 12 aa, Artificial sequence
  complementarity-determining region (CDR) H3
ASDYYGSGSFNY

SEQ ID NO: 105 - protein, 12 aa, Artificial sequence
  complementarity-determining region (CDR) H3
ASDYYGSGALEY

SEQ ID NO: 106 - protein, 12 aa, Artificial sequence
  complementarity-determining region (CDR) H3
ASDYYGSGSLEY

SEQ ID NO: 107 - protein, 12 aa, Artificial sequence
  complementarity-determining region (CDR) H3
ASDYYGSGSYQY

SEQ ID NO: 108 - protein, 12 aa, Artificial sequence
  complementarity-determining region (CDR) H3
ASDYYGSGALQY

SEQ ID NO: 109 - protein, 12 aa, Artificial sequence
  complementarity-determining region (CDR) H3
ASDYYGSGSLAY

SEQ ID NO: 110 - protein, 12 aa, Artificial sequence
  complementarity-determining region (CDR) H3
ASDYYGSGAFQY

SEQ ID NO: 111 - protein, 12 aa, Artificial sequence
  complementarity-determining region (CDR) H3
ASDYYGSGSFQY

SEQ ID NO: 112 - protein, 12 aa, Artificial sequence
  complementarity-determining region (CDR) H3
ASDYYGSGSLAY

SEQ ID NO: 113 - protein, 12 aa, Artificial sequence
  complementarity-determining region (CDR) H3
ASDYYGSGAYEY

SEQ ID NO: 114 - protein, 12 aa, Artificial sequence
  complementarity-determining region (CDR) H3
ASDYYGSGSYQY

SEQ ID NO: 115 - protein, 12 aa, Artificial sequence
  complementarity-determining region (CDR) H3
ASDYYGSGSYQY

SEQ ID NO: 116 - protein, 12 aa, Artificial sequence
  complementarity-determining region (CDR) H3
ASDYYGSGALAY

SEQ ID NO: 117 - protein, 12 aa, Artificial sequence
  complementarity-determining region (CDR) H3
ASDYYGSGALAY

SEQ ID NO: 118 - protein, 12 aa, Artificial sequence
  complementarity-determining region (CDR) H3
ASDYYGSGSYAY

SEQ ID NO: 119 - protein, 9 aa, Artificial sequence
  complementarity-determining region (CDR) L3
QQHYSIPFT

SEQ ID NO: 120 - protein, 7 aa, Artificial sequence
  loop hypothetical amino acid sequence
APTESVV